(19) United States
(12) Patent Application Publication
(10) Pub. No.: US 2018/0014731 A1
(43) Pub. Date: Jan. 18, 2018

(54) BIOMETRIC INFORMATION MEASURING APPARATUS AND NON-TRANSITORY COMPUTER READABLE STORAGE MEDIUM

(71) Applicant: FUJI XEROX CO., LTD., Tokyo (JP)

(72) Inventors: Tsutomu OTSUKA, Kanagawa (JP); Shingo SUDA, Kanagawa (JP); Kazuhiro SAKAI, Kanagawa (JP); Manabu AKAMATSU, Kanagawa (JP)

(73) Assignee: FUJI XEROX CO., LTD., Tokyo (JP)

(21) Appl. No.: 15/439,300

(22) Filed: Feb. 22, 2017

(30) Foreign Application Priority Data
Jul. 15, 2016 (JP) 2016-140622

(51) Int. Cl. A61B 5/00; A61B 5/02

(52) U.S. Cl. CPC A61B 5/0082; A61B 5/02007; A61B 5/74

(57) ABSTRACT

A biometric information measuring apparatus includes a light emitting unit, a light receiving unit, a detecting unit, and a controller. The light emitting unit is configured to emit light. The light receiving unit is configured to receive light. The detecting unit is configured to detect a frequency distribution of the light received by the light receiving unit. When a feature which is obtained in response to a living body being irradiated with light is no longer included in a frequency component detected by the detecting unit during a period in which biometric information is measured using the frequency distribution detected by the detecting unit, the controller stops measurement of biometric information in the living body.

### CROSS-REFERENCE TO RELATED APPLICATIONS

This application is based on and claims priority under 35 USC 119 from Japanese Patent Application No. 2016-140622 filed Jul. 15, 2016.

### BACKGROUND

### Technical Field

The present invention relates to a biometric information measuring apparatus and a non-transitory computer readable storage medium.

### SUMMARY

According to an aspect of the invention, a biometric information measuring apparatus includes a light emitting unit, a light receiving unit, a detecting unit, and a controller. The light emitting unit is configured to emit light. The light receiving unit is configured to receive light. The detecting unit is configured to detect a frequency distribution of the light received by the light receiving unit. When a feature which is obtained in response to a living body being irradiated with light is no longer included in a frequency component detected by the detecting unit during a period in which biometric information is measured using the frequency distribution detected by the detecting unit, the controller stops measurement of biometric information in the living body.

### BRIEF DESCRIPTION OF THE DRAWINGS

Exemplary embodiments of the present invention will be described in detail based on the following figures, wherein:

FIG. 1 is a view illustrating a configuration example of a biometric information measuring apparatus according to a first exemplary embodiment;

FIG. 2 is a view illustrating an example of arrangement of a light emitting element and a light receiving element;

FIG. 3 is a view illustrating an example of a change in a received light intensity with respect to reflected light from a living body;

FIG. 4 is a schematic view used to explain a Doppler shift that occurs when a blood vessel is irradiated with a laser beam;

FIG. 5 is a schematic view used to explain a speckle generated when a blood vessel is irradiated with a laser beam;

FIG. 6 is a view illustrating an example of a spectral distribution of light reflected by a living body;

FIG. 7 is a graph illustrating an example of a change in a blood flow rate;

FIG. 8 is a view illustrating a configuration example of a main part of an electric system of the biometric information measuring apparatus according to the first exemplary embodiment;

FIG. 9 is a flowchart illustrating an example of a flow of a biometric information measuring process according to the first exemplary embodiment;

FIG. 10 is a view for explaining a spectral distribution of light transmitted through or reflected by a living body and characteristics of a spectral distribution of external light;

FIG. 11 is a view illustrating an example of an emission pattern of a light emitting element in a standby mode and a measurement mode;

FIG. 12A is a view illustrating an example of an emission state of a light emitting element in an emission period;

FIG. 12B is a view illustrating an example of an emission state of a light emitting element in an emission period;

FIG. 13 is a view illustrating an example of an emission pattern of a light emitting element in a standby mode and a measurement mode;

FIG. 14 is a view illustrating an example of an emission pattern of a light emitting element in a standby mode and a measurement mode;

FIG. 15 is a flowchart illustrating a modification example of the biometric information measuring process according to the first exemplary embodiment;

FIG. 16 is a flowchart illustrating a modification example of the biometric information measuring process according to the first exemplary embodiment;

FIG. 17 is a view illustrating an example of an emission pattern of a light emitting element in a standby mode and a measurement mode in a modification example of the biometric information measuring process according to the first exemplary embodiment;

FIG. 18 is a view illustrating a configuration example of a biometric information measuring apparatus according to a second exemplary embodiment;

FIG. 19 is a graph illustrating an example of a change in the quantity of light absorbed by a living body;

FIG. 20 is a view illustrating a configuration example of a main part of an electric system of the biometric information measuring apparatus according to the second exemplary embodiment;

FIG. 21 is a flowchart illustrating an example of a flow of a biometric information measuring process according to the second exemplary embodiment;

FIG. 22 is a view illustrating an example of an emission pattern of a light emitting element in a standby mode and a measurement mode in the biometric information measuring process according to the second exemplary embodiment;

FIG. 23 is a view illustrating a configuration example of a biometric information measuring apparatus according to a third exemplary embodiment;

FIG. 24 is a view illustrating a configuration example of a main part of an electrical system of the biometric information measuring apparatus according to the third exemplary embodiment; and

FIG. 25 is a flowchart illustrating an example of a flow of a biometric information measuring process according to the third exemplary embodiment.

### DETAILED DESCRIPTION

Hereinafter, exemplary embodiments of the present invention will be described in detail with reference to the drawings. Throughout the drawings, elements having actions or functions in charge of the same work are denoted by the same reference numerals and explanation of which will not be repeated.

### First Exemplary Embodiment

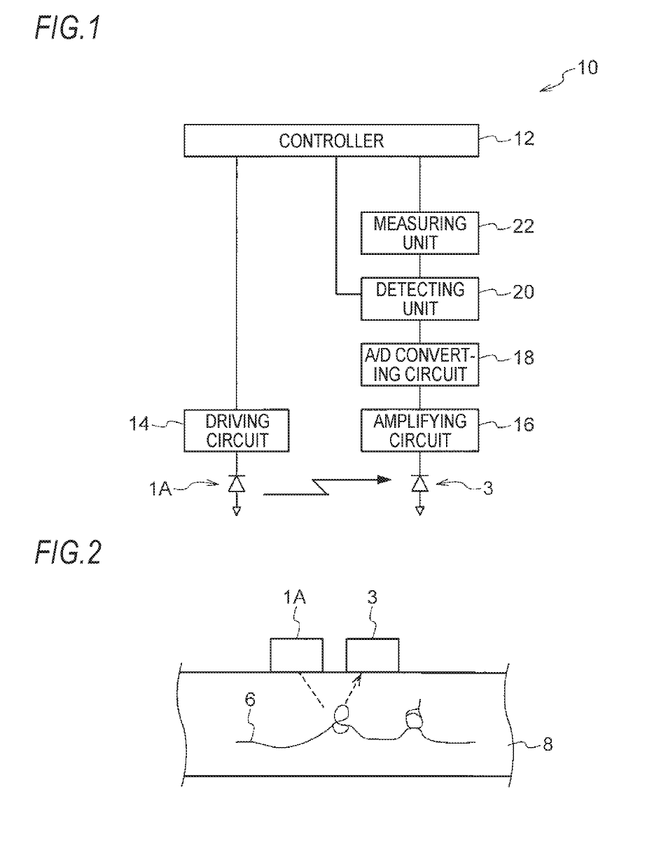

First, FIG. 1 illustrates a configuration example of a biometric information measuring apparatus 10 according to a first exemplary embodiment. As illustrated in FIG. 1, the biometric information measuring apparatus 10 includes alight emitting element 1A, alight receiving element 3, a controller 12, a driving circuit 14, an amplifying circuit 16, an A/D converting circuit 18, a detecting unit 20 and a measuring unit 22 and measures a blood flow rate, which is an example of biometric information, at a portion such as a fingertip, a wrist, an earlobe or the like.

The light emitting element 1A is an element that emits coherent light having coherency with uniform phases, more specifically, laser light. Although only one light emitting element 1A is illustrated in FIG. 1, plural light emitting elements 1A may be used. In addition, the light emitting element 1A may be a surface emitting laser element or an edge emitting laser element. Hereinafter, a “laser beam” may be simply referred to as “light” and, particularly when it is desirable to emphasize that the light is laser light, it is expressed as “laser beam” as it is.

The driving circuit 14 supplies, for example, power for driving the light emitting element 1A according to an instruction of the controller 12 to be described later, and drives the light emitting element 1A so that the light emitting element 1A emits light or stops the emission.

The light receiving element 3 receives light emitted from the light emitting element 1A, or external light around the biometric information measuring apparatus 10, which is emitted from the sun, a lighting fixture or the like, and converts the received light into a physical quantity corresponding to the intensity of the received light. Here, as an example, descriptions will be made on the assumption that the light receiving element 3 outputs a voltage corresponding to the intensity of the received light, but the light receiving element 3 may output a current according to the intensity of the received light, or may change a resistance.

The amplifying circuit 16 amplifies the voltage corresponding to the intensity of the light received by the light receiving element 3 to a voltage level defined as an input voltage range of the A/D converting circuit 18.

The A/D converting circuit 18 receives the voltage amplified by the amplifying circuit 16 as an input, digitizes the intensity of the light received by the light receiving element 3, which is represented by the magnitude of the voltage, and outputs the digitized light intensity to the detecting unit 20.

The detecting unit 20 performs a fast Fourier transform (FFT) for a temporal change in the light intensity digitized by the A/D converting circuit 18 every predetermined process time (sampling time), and detects a frequency distribution (spectral distribution) for each frequency ω. Here, the sampling time is, for example, about several milliseconds to several hundred milliseconds. As an example, the sampling time is set to 20 ms.

The controller 12 receives various instructions from a user and determines from the spectral distribution detected by the detecting unit 20 whether or not light transmitted through a blood vessel of the living body or light reflected from the blood vessel of the living body has been received by the light receiving element 3. When it is determined that the light transmitted through the blood vessel of the living body or the light reflected by the blood vessel of the living body has been received by the light receiving element 3, the controller 12 shifts an operation state of the biometric information measuring apparatus 10 from a standby mode (standby state) to a measurement mode (measurement state). As an example, based on the spectral distribution detected by the detecting unit 20, the controller 12 controls the driving circuit 14 and the measuring unit 22 to start measurement of a blood flow rate, and shifts the biometric information measuring apparatus 10 to a measurement state (i.e., measurement mode) of biometric information.

Meanwhile, in a state where the biometric information measuring apparatus 10 is already in the measurement mode, upon receiving a measurement end instruction from the user, the controller 12 controls the driving circuit 14 and the measuring unit 22 to stop the measurement of the blood flow rate.

Further, if the biometric information measuring apparatus 10 is already in the measurement mode and if it is determined that the light transmitted through the blood vessel of the living body or the light reflected by the blood vessel of the living body has no longer been received by the light receiving element 3, the controller 12 controls the driving circuit 14 and the measuring unit 22 to stop the measurement of the blood flow rate without receiving the measurement end instruction from the user.

According to an instruction from the controller 12, the measuring unit 22 measures the blood flow rate based on the spectral distribution detected by the detecting unit 20.

The “standby mode” used herein is a mode at a stage before the transition to the measurement mode or a later mode at a stage after the end of the measurement mode, and refers to a state in which the quantity of light emitted from the light emitting element 1A is reduced, a state in which some functions of the biometric information measuring apparatus 10 are not operated, etc., as compared with the measurement mode. The standby mode also includes a preparatory measurement state for detecting biometric information in order to shift to the measurement mode. Meanwhile, the “measurement mode” used herein is a mode for measuring biometric information and is also a mode for performing measurement for reporting a result to the user. The measurement mode does not include a preparatory measurement state for shifting to the measurement mode.

Next, the principle of measurement of the blood flow rate in the biometric information measuring apparatus 10 will be described. The biometric information measuring apparatus measures the biometric information using the light transmitted through the blood vessel of the living body or the light reflected by the blood vessel of the living body. In the case of measuring the biometric information using the light transmitted through the blood vessel, the light emitting element 1A and the light receiving element 3 are arranged to face each other with a living body such as a fingertip being sandwiched therebetween. Meanwhile, in the case of measuring the biometric information using the light reflected by the blood vessel, the light emitting element 1A and the light receiving element 3 are arranged side by side along the surface of the living body. It is possible to measure the blood flow rate of the blood flowing through the blood vessel on the same principle by using either the light transmitted through the blood vessel of the living body or the light reflected by the blood vessel of the living body. Accordingly, as an example, a case where the light reflected by the blood vessel of the living body is used to measure the blood flow rate will be described below.

FIG. 2 is a view illustrating an example of arrangement of the light emitting element 1A and the light receiving element 3 in the biometric information measuring apparatus 10. In the case of measuring the blood flow rate using the light (reflected light) reflected by the blood vessel of the living body, the light emitting element 1A and the light receiving element 3 are arranged side by side along the surface of a living body 8. In this case, the light receiving element 3 receives the light of the light emitting element 1A reflected by a blood vessel 6 of the living body 8.

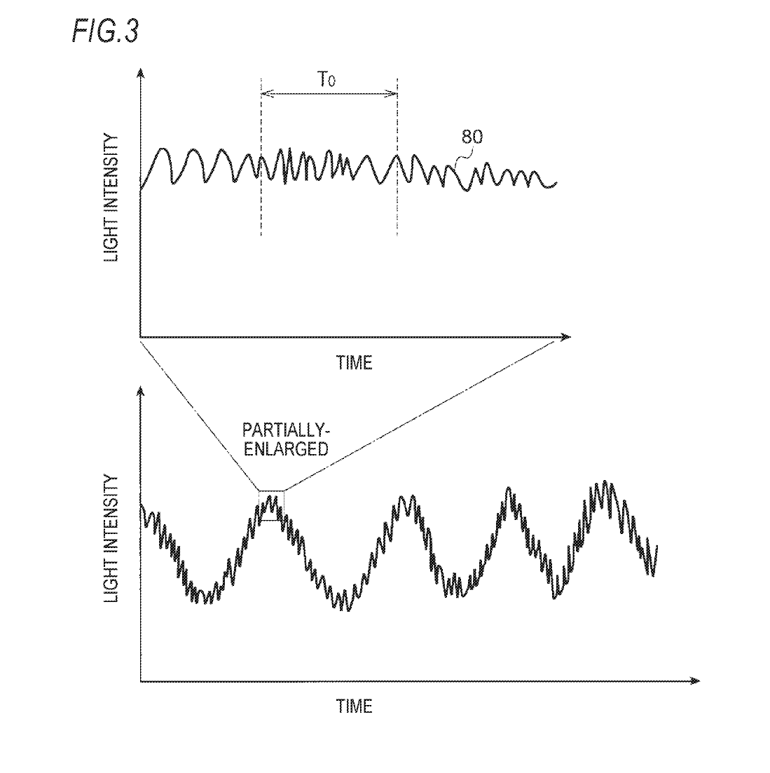

FIG. 3 is an example of a graph 80 illustrating the intensity of the reflected light of the light emitting element 1A, which is received by the light receiving element 3. In the graph 80 of FIG. 3, a horizontal axis represents the lapse of time and a vertical axis represents the output of the light receiving element 3, that is, the intensity (received light intensity) of the light received by the light receiving element 3.

As illustrated in FIG. 3, the received light intensity of the light receiving element 3 varies with the lapse of time. This is believed to be due to the influence of three optical phenomena appearing when the living body 8 including the blood vessel 6 is irradiated with light.

The first optical phenomenon may be a change in absorption of light due to a change in the volume of the blood present in the blood vessel 6 being measured, due to a pulsation. Since the blood contains hematopoietic cells such as, for example, red blood cells and moves through the blood vessel 6 such as a capillary blood vessel, the number of hematopoietic cells moving through the blood vessel is varied with a change in the volume of blood, which may affect the received light intensity in the light receiving element 3.

The second optical phenomenon may be an influence by a Doppler shift.

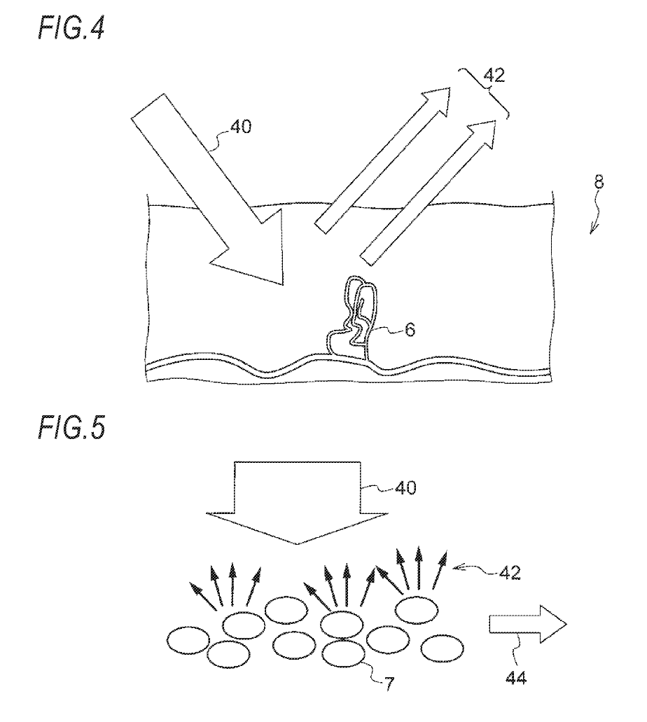

As illustrated in FIG. 4, when coherent light 40 having a frequency ω₀, such as, for example, a laser beam, is emitted from the light emitting element 1A onto a region including the blood vessel 6, scattered light 42 scattered by the hematopoietic cells moving through the blood vessel 6 results in a Doppler shift having a difference frequency Δω₀ determined depending on a movement speed of the hematopoietic cells. Meanwhile, scattered light 42 scattered by a tissue (stationary tissue) such as a skin which does not include a moving body such as the hematopoietic cells maintains the same frequency ω₀ as the emitted laser beam. Therefore, the frequency ω₀+Δω₀ of the laser beam scattered by the blood vessel 6 and the frequency ω₀ of the laser beam scattered by the stationary tissue interfere with each other, a beat signal having the difference frequency Δω₀ is observed in the light receiving element 3, and the received light intensity of the light receiving element 3 is changed with the lapse of time. The difference frequency Δω₀ of the beat signal observed in the light receiving element 3 depends on the movement speed of the hematopoietic cells and is included in a range with the upper limit of approximately several tens of kHz.

The third optical phenomenon may be an effect by a speckle.

As illustrated in FIG. 5, when coherent light 40 such as a laser beam is emitted from the light emitting element 1A to the hematopoietic cells 7 such as red blood cells that move through the blood vessel 6 in the direction of an arrow 44, the laser beam hitting the hematopoietic cells 7 is scattered in different directions. The scattered lights have different phases and therefore interfere randomly with each other. This forms a random-speckled light intensity distribution. Alight intensity distribution pattern thus formed is called a “speckle pattern.”

As described previously, since the hematopoietic cells 7 move through the blood vessel, a light scattering state in the hematopoietic cells 7 is changed and the speckle pattern is changed with the lapse of time. Therefore, the received light intensity of the light receiving element 3 is varied with the lapse of time.

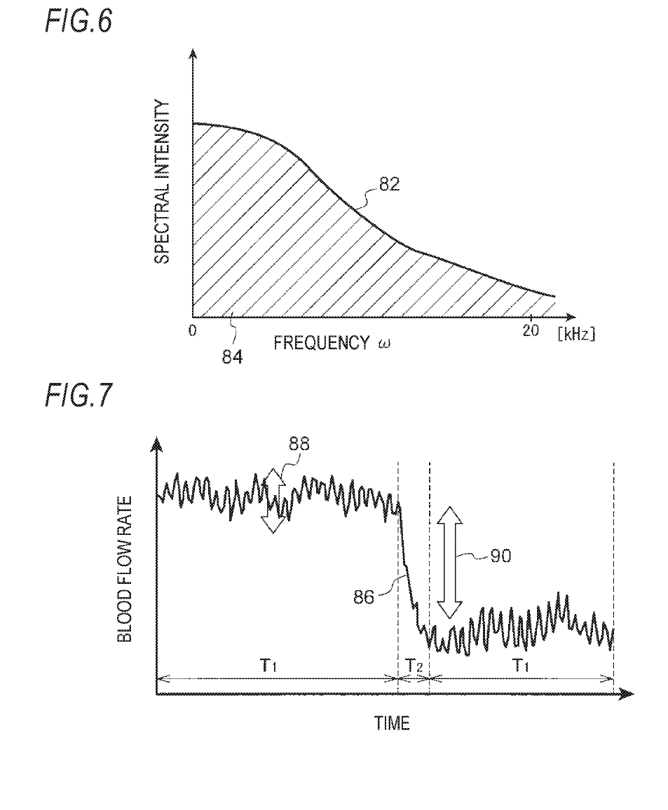

In this way, when the time-variable received light intensity of the light receiving element 3 is obtained, data included in the range of the predetermined unit time T₀ is cut out and, for example, FFT processing is executed on the data to thereby obtain a spectral distribution for each frequency co. FIG. 6 illustrates an example of a spectral distribution 82 of light reflected by the blood vessel 6 for each frequency co in the unit time T₀. In the spectral distribution 82 in FIG. 6, a horizontal axis represents the frequency ω and a vertical axis represents the magnitude of a frequency component for each frequency ω, that is, the spectral intensity. The spectral distribution 82 of light reflected by the blood vessel 6 appears over a range from 0 Hz to about several tens of kHz, specifically from 0 Hz to about 20 kHz.

Here, the blood volume is proportional to a value obtained by normalizing the area indicated by a shaded region 84 surrounded by the spectral distribution 82, a frequency coordinate axis and a spectral intensity coordinate axis with the total light quantity. In addition, since a velocity (blood velocity) of blood flowing through the blood vessel 6, which is an example of the biometric information, is proportional to a frequency average value of the spectral distribution 82, the blood velocity is proportional to a value obtained by dividing a value, which is obtained by integrating the product of the frequency ω and the spectral intensity at the frequency ω with respect to the frequency ω, by the area of the shaded region 84.

Meanwhile, the blood flow rate is expressed by the product of the blood volume and the blood velocity, and thus may be calculated from a measured blood volume and a measured blood velocity.

FIG. 7 is an example of a graph 86 illustrating a change in a blood flow rate per unit time T₀, which is measured as described above. In the graph 86 in FIG. 7, a horizontal axis represents time and a vertical axis represents a blood flow rate.

As illustrated in FIG. 7, the blood flow rate is varied with time and the tendency of the variation is classified into two types. For example, in FIG. 7, a variation range 90 of the blood flow rate in an interval T₂ is larger than a variation range 88 of the blood flow rate in an interval T₁. It is believed that this is because a change in the blood flow rate in the interval T₁ is mainly a change in the blood flow rate according to the movement of a pulse while a change in the blood flow rate in the interval T₂ is a change in the blood flow rate caused by, for example, a congestion or the like.

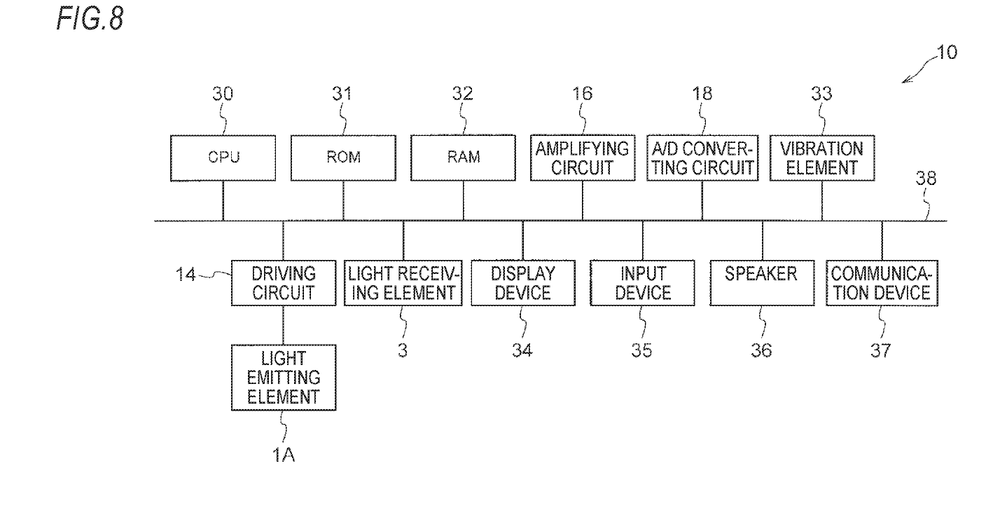

Next, a configuration of a main part of an electric system of the biometric information measuring apparatus 10 according to the first exemplary embodiment will be described with reference to FIG. 8. Hereinafter, descriptions will be given with the presumption that the biometric information measuring apparatus 10 according to the present invention is incorporated in a portable terminal such as a smartphone or the like. However, this is just an example and it goes without saying that the biometric information measuring apparatus 10 may be incorporated in a device other than the portable terminal or may be configured as a single device.

As illustrated in FIG. 8, the biometric information measuring apparatus 10 according to the first exemplary embodiment includes a detecting unit for detecting the spectral distribution of the light received by the light receiving element 3, a measuring unit for measuring the blood flow rate, and a central processing unit (CPU) 30 as an example of a controller for controlling the driving circuit 14 for driving the light emitting element 1A, the detecting unit and the measuring unit. Further, the biometric information measuring apparatus 10 includes a read only memory (ROM) 31 in which various programs, various parameters and the like are stored in advance, and a random access memory (RAM) 32 used as a work area or the like when the CPU 30 executes the various programs.

The CPU 30, the ROM 31 and the RAM 32 are interconnected via an internal bus 38 of the biometric information measuring apparatus 10. In addition, the driving circuit 14, the light receiving element 3, the amplifying circuit 16, the A/D converting circuit 18, a vibration element 33, a display device 34, an input device 35, a speaker 36 and a communication device 37 are connected to the internal bus 38. In addition, the light emitting element 1A is connected to the driving circuit 14.

Among these, the vibration element 33 is an element for notifying, in vibration, a user of information on the measurement of the biometric information, such as for notification of start and end of measurement of the blood flow rate. For example, a vibration motor or the like may be used as the vibration element 33. When the biometric information measuring apparatus 10 is incorporated in a smartphone, the biometric information measuring apparatus 10 may use a vibrator of the smartphone as the vibration element 33.

The display device 34 is a device for visually notifying the user of information on measurement of the biometric information, such as for notification of start and end of measurement of the blood flow rate or notification of a measured blood flow rate. For example, a liquid crystal display, an organic EL or the like may be used as the display device 34. When the biometric information measuring apparatus is incorporated in the smartphone, the biometric information measuring apparatus 10 may use a display panel of the smartphone as the display device 34. In addition, the display device 34 may be configured with light emitting elements such as LEDs, so that the number, shape, color, etc. of LEDs to be turned ON may be changed to notify to a user.

The input device 35 is a device for receiving an instruction from the user to the biometric information measuring apparatus 10. For example, a button, a touch panel or the like may be used as the input device 35. A microphone for converting a vocal instruction from a user into an electric signal is also an example of the input device 35. When the biometric information measuring apparatus 10 is incorporated in the smartphone, the biometric information measuring apparatus 10 may use the touch panel, the button, the microphone, etc. incorporated in the display panel of the smartphone as the input device 35.

The speaker 36 is a device for notifying, by voice, the user of information on measurement of the biometric information, such as notification of start and end of measurement of the blood flow rate or notification of a measured blood flow rate. For example, an acoustic device incorporating the speaker 36, such as a headphone or an earphone, may be an example of the speaker 36. When the biometric information measuring apparatus 10 is incorporated in the smartphone, the biometric information measuring apparatus 10 may use, for example, a speaker 36 incorporated in the smartphone.

The communication device 37 is a device provided with a communication protocol for exchanging data with other devices connected to a network such as the Internet. For example, the communication device 37 may transmit a measured blood flow rate to another device or may receive a program of the biometric information measuring apparatus 10 from another device. When the biometric information measuring apparatus is incorporated in the smartphone, the biometric information measuring apparatus 10 may use, for example, a communication device 37 incorporated in the smartphone. It should be noted here that the communication device 37 may be either wired to a network or wirelessly connected to a network.

In addition, the CPU 30 incorporates a timer for measuring the elapsed time from a designated time point.

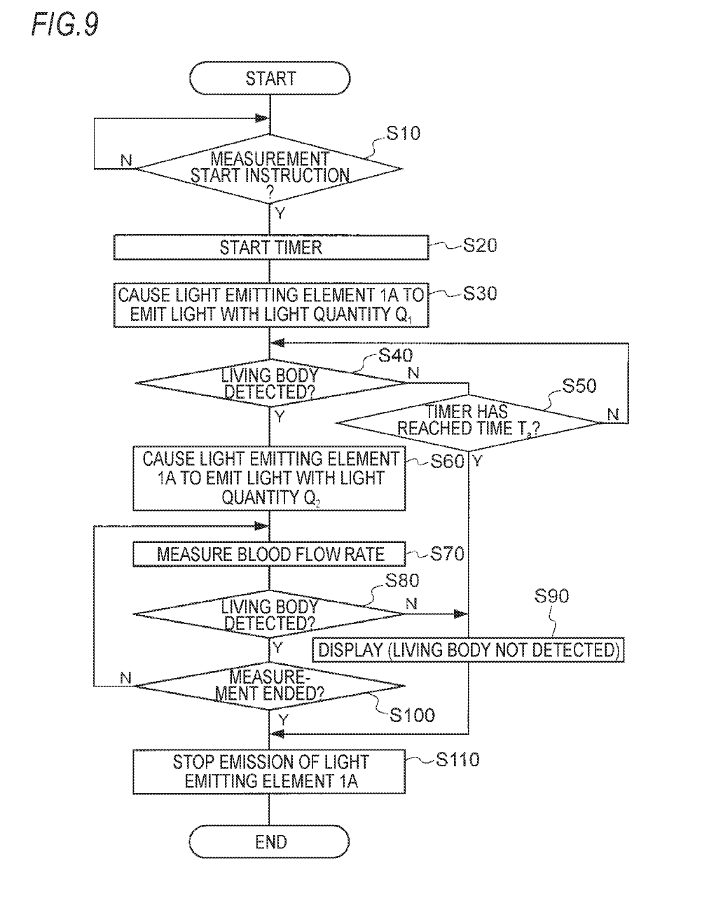

Next, the operation of the biometric information measuring apparatus 10 will be described. FIG. 9 is a flowchart illustrating an example of a flow of a biometric information measuring process executed by the CPU 30 when a smartphone in which the biometric information measuring apparatus 10 is incorporated is powered on.

A program (biometric information measuring program) for defining the biometric information measuring process is installed in advance in the ROM 31, for example. At the point of start of the biometric information measuring program, the light emitting element 1A is in an emission stop state where no light is emitted.

First, at the step S10, the CPU 30 determines whether or not a blood flow rate measurement start instruction has been received from a user. The blood flow rate measurement start instruction is notified to the CPU 30, for example when the user presses a button (measurement start button) for starting measurement of the blood flow rate, which is displayed on the display device 34 on which a touch panel is superimposed. Meanwhile, the blood flow rate measurement start instruction is not limited thereto but may be an instruction to start blood flow rate measurement software by the user. Further, for example, the user may issue a vocal instruction to start the blood flow rate measurement.

When there is no measurement start instruction from the user, the process in step S10 is repeatedly performed to wait for a measurement start instruction. Meanwhile, when a measurement start instruction is received, the process proceeds to the step S20.

At the step S20, the CPU 30 starts the timer incorporated in the CPU 30.

At the step S30, the CPU 30 controls the driving circuit 14 to cause the light emitting element 1A to emit light with the light quantity Q₁. The term “light quantity” used herein refers to a physical quantity (in the unit of [1 m˜s]) represented by the product of the intensity (flux) of light and time when the light is emitted from a light source such as the light emitting element 1A to the space. Therefore, even when the light emitting element 1A is caused to emit light with a predetermined light intensity, the light quantity of the light emitting element 1A increases with an increase in an emission period.

In addition, the light quantity Q₁ is set to a light quantity sufficient to detect the spectral distribution 82 required to detect the living body 8 at the step S40 to be described later. A specific value of the light quantity Q₁ is determined by experiments performed by the biometric information measuring apparatus 10 as an actual apparatus or a computer simulation based on design specifications of the biometric information measuring apparatus 10.

At the step S40, the CPU 30 performs FFT processing on a temporal change in the light intensity digitized by the A/D converting circuit 18 and detects the spectral intensity corresponding to plural frequencies ω as the spectral distribution 82. Then, the CPU 30 determines whether or not the spectral intensity at a predetermined frequency (reference frequency) is larger than a threshold value set in advance as a value obtained in response to the living body 8 being irradiated with light from the light emitting element 1A. In addition, only the spectral intensity at one reference frequency may be detected instead of the spectral intensity corresponding to the plural frequencies ω.

When the spectral intensity at the reference frequency is equal to or less than the threshold value, that is, when the living body 8 cannot be detected at a position (measurement position) facing the emission surface of the light emitting element 1A, the process proceeds to the step S50. Meanwhile, when the spectral intensity at the reference frequency is larger than the threshold value, that is, when the living body is detected at the measurement position, the process proceeds to the step S60.

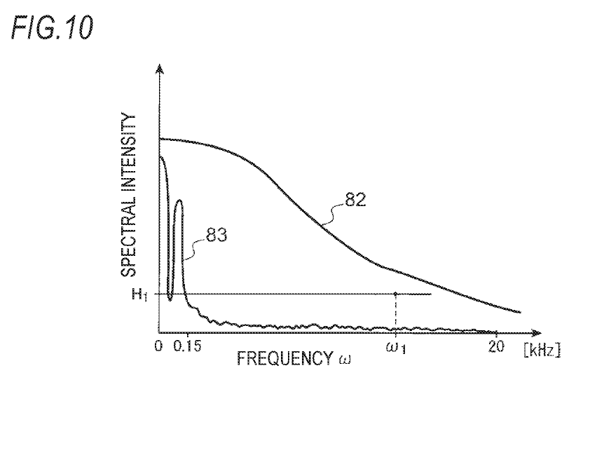

The threshold value of the spectral intensity used for detection of the living body 8 will now be described with reference to FIG. 10. As described above, the spectral distribution 82 of the light of the light emitting element 1A reflected by the living body 8 appears over the range of 0 Hz to about 20 kHz. In the case of the light of the light emitting element 1A reflected by the living body 8, the spectral distribution 82 ranging from 0 Hz to about 20 kHz has the lowest spectral intensity below which the spectral intensity does not decrease for each frequency.

Therefore, by setting a specific frequency ω₁ as the reference frequency and setting the lowest spectral intensity at the reference frequency ω₁ to a threshold value H₁, when the spectrum intensity at the reference frequency ω₁ is larger than the threshold value H₁, it can be determined that the living body 8 is placed at the measurement position.

Here, the reference frequency ω₁ may be set to any frequency as long as it ranges from 0 Hz to about 20 kHz.

However, when external light of a lighting fixture or the like is received by the light receiving element 3, the reference frequency ω₁ may be set in consideration of a spectral distribution 83 of the external light. This is because, in a case where the reference frequency ω₁ is set to a frequency band where the spectral intensity in the external light is strong, it may be erroneously determined that the living body 8 is placed although the living body 8 is not present, depending on a relationship between the threshold value H₁ and the intensity of external light. Therefore, it is desirable to set the reference frequency ω₁ while avoiding frequencies which are likely to be affected by the external light, specifically frequencies of about 100 Hz and about 120 Hz which are twice the commercial frequency emitted by an incandescent lamp or the like. More specifically, it is more desirable to set the reference frequency ω₁ in a range of about 150 Hz to about 20 kHz.

In addition, since the threshold value H₁ at the reference frequency ω₁ is also varied depending on the light intensity of the light emitting element 1A that is caused to emit light at the step S30, the threshold value H₁ is determined by experiments performed by the biometric information measuring apparatus 10 as an actual apparatus or a computer simulation based on the design specifications of the biometric information measuring apparatus 10 and is stored in advance in the ROM 31, for example.

At the step S40, it is determined that the living body 8 has been detected when the spectral intensity of the preset reference frequency ω₁ is larger than the threshold value H₁. However, the method for detecting the living body 8 is not limited thereto.

For example, when the area of the shaded region 84 surrounded by the spectral distribution 82, the frequency coordinate axis, and the spectral intensity coordinate axis illustrated in FIG. 6 is equal to or larger than a predetermined size, it may be determined that the living body 8 has been detected. Alternatively, when plural different reference frequencies are set and the spectral intensities of the respective reference frequencies are larger than the respective threshold values set for the respective reference frequencies, it may be determined that the living body 8 has been detected. In this case, the threshold values set for the respective reference frequencies may be set to the same value.

In addition, the spectral intensity at the reference frequency may be measured plural times and it may be determined that the living body 8 has been detected when the spectral intensity continuously exceeds the threshold value plural times. Furthermore, when plural different reference frequencies are set and the average value of the spectral intensities of the respective reference frequencies is larger than the threshold value, it may be determined that the living body 8 has been detected.

When the orientation of a portable terminal such as a smartphone in which the biometric information measuring apparatus 10 is incorporated is varied according to the motion of the user or the like, the quantity of light received by the light receiving element 3 may be varied and an intensive spectrum at a specific frequency corresponding to the variation may be detected, which may lead to erroneous detection in some cases. Therefore, when the spectral intensities at plural reference frequencies are compared with the respective corresponding threshold values or the spectral intensities are measured plural times, the detection accuracy of the living body 8 is improved as compared with a case where the spectral intensity at one measurement using one reference frequency ω₁ is compared with the threshold value H₁.

At the step S50 to which the process proceeds when it is determined at the step S40 that the living body 8 cannot be detected, the CPU 30 determines whether or not the elapsed time of the timer started at the step S20 is equal to or longer than time Tₐ. The time Tₐ is a value that defines a detection period of the living body 8 at the step S40. When the elapsed time of the timer is less than the time Tₐ, the process proceeds to the step S40 where the determination on whether or not the living body 8 has been detected is repeated.

Meanwhile, when the elapsed time of the timer is equal to or longer than the time Tₐ, the process proceeds to the step S90 where the detection of the living body 8 is stopped.

In this way, the time Tₐ has a role of avoiding a situation in which when the living body 8 is not detected at the step S40, the step S40 is indefinitely executed, so that the following process is not performed.

At the step S60 to which the process proceeds when it is determined at the step S40 that the living body 8 has been detected, the CPU 30 controls the driving circuit 14 to cause the light emitting element 1A to emit light with the light quantity Q₂. The light quantity Q₂ used herein refers to a light quantity larger than the light quantity Q₁ of light emitted by the light emitting element 1A at the step S30.

The light quantities Q₁ and Q₂ are both set to a light quantity at which the spectral distribution 82 of the light reflected by the blood vessel 6 may be obtained. Specific values of the light quantities Q₁ and Q₂ are determined by experiments performed by the biometric information measuring apparatus 10 as an actual apparatus or a computer simulation based on design specifications of the biometric information measuring apparatus 10.

At the step S70, in a state where the light quantity of the light emitting element 1A is set to the light quantity Q₂, the CPU 30 performs FFT processing on the temporal change in the light intensity digitized by the A/D converting circuit 18 to thereby detect the spectral distribution 82 for each frequency ω. The CPU 30 uses the detected spectral distribution 82 to calculate the blood volume and the blood velocity in accordance with the previously-described method, measures the product of the blood volume and the blood velocity as the blood flow rate, and stores a result of the measurement in the RAM 32, for example.

In this case, the CPU 30 may cause the display device 34 to display the measurement result of the blood flow rate according to a displaying method such as numerical values, graphs, characters or the like through which the user may recognize the measurement result. Further, the CPU 30 may transmit the measurement result of the blood flow rate to another device connected to a network via the communication device 37 so that the measurement result may be stored and displayed in another device.

At the step S80, the CPU 30 performs the same process as the step S40 to determine, based on a result of the comparison between the reference frequency ω₁ and the threshold value H₁, whether or not the living body 8 has been detected. In addition, at the step S80, it may be determined, based on a reference frequency and a threshold value different respectively from the reference frequency ω₁ and the threshold value H₁ used at the step S40, whether or not the living body 8 has been detected.

The reason why the living body 8 is again detected at the step S80 is that, in a situation where the biometric information measuring apparatus 10 is in the measurement mode in which the measurement of the blood flow rate is started at the step S70, when the user releases the living body 8 such as a finger from the measurement position, the blood flow rate may not be measured correctly in some cases.

Therefore, when it is determined at the step S80 that the living body 8 cannot be detected, the process proceeds to the step S90 as in the case where it is determined at the step S50 that the elapsed time of the timer becomes equal to or longer than the time Tₐ. At this case, the determination at the step S80 may be performed simultaneously with the step S70 using the measurement result at the step S70. That is, it may be determined, based on the comparison result between the reference frequency ω₁ and the threshold value H₁ at the step S70, whether or not the user's finger or the like is away from the measurement position.

At the step S90, the CPU 30 displays a message such as “The living body cannot be detected” or the like on, for example, the display device 34 to notify the user that the living body 8 has left the measurement position. Incidentally, the above notification is not limited to the displaying on the display device 34 but the user may be notified by, for example, outputting a voice from the speaker 36 or vibrating the vibration element 33.

After the step S90, at the step S110, the CPU 30 controls the driving circuit 14 to stop the emission in the light emitting element 1A to stop the measurement of the blood flow rate.

Meanwhile, when it is determined at the step S80 that the living body 8 has been detected, the process proceeds to the step S100.

At the step S100, it is determined whether or not to end the measurement. For example, the CPU 30 determines whether or not a measurement end instruction to end the measurement of the blood flow rate has been received from the user. The blood flow rate measurement end instruction is notified to the CPU 30, for example when the user presses a button (measurement end button) for stopping the measurement of the blood flow rate, which is displayed on the display device 34 on which a touch panel is superimposed. Note that the blood flow rate measurement end instruction is not limited thereto but the user may issue the instruction by voice, for example. In addition, the measurement may be terminated when a predetermined measurement time has elapsed or acquisition of information necessary for measurement is completed.

When the determination process at the step S100 is negative, for example, when the measurement end instruction has not been received from the user, the process proceeds to the step S70 and repeats the steps S70, S80 and S100 to continue to measure the blood flow rate until the measurement end instruction is received from the user or the living body 8 is no longer detected at the measurement position.

Meanwhile, when the determination process at the step S100 is affirmative, that is, when the measurement end instruction has been received from the user, the process proceeds to the step S110.

Then, at the step S110, the CPU 30 controls the driving circuit 14 to stop the emission in the light emitting element 1A to stop the measurement of the blood flow rate.

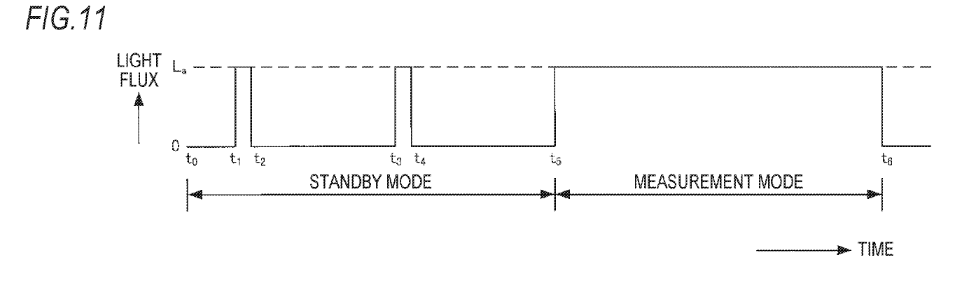

FIG. 11 is a view illustrating an example of the emission state of the light emitting element 1A when the biometric information measuring process illustrated in FIG. 9 is performed.

As illustrated in FIG. 11, in the standby mode represented by a period from time t₀ to time t₅, the biometric information measuring apparatus 10 drives the light emitting element 1A with such an emission pattern of a cycle of 200 ms that light is emitted with a light flux Lₐ only for the period of 20 ms and then the emission is stopped in the next period of 180 ms, thereby setting the light quantity emitted from the light emitting element 1A to the light quantity Q₁. Here, when it is tried to determine the presence or absence of the living body 8 based on, for example, a pulse or the like, it usually takes a time (several seconds) for several beats. However, in this exemplary embodiment, since the presence or absence of the living body 8 is determined based on the result of comparison between the spectral intensity at the reference frequency ω₁ with the threshold value H₁, the presence or absence of the living body is determined when the light emitting element 1A emits light only for a period required to detect the spectral intensity, for example, for a period of several ms to several hundred ms.

Note that the emission pattern of the light emitting element 1A in the standby mode is not limited thereto. For example, the emission period of the light emitting element 1A in the standby mode may be set in accordance with the process time of the FFT processing in the detecting unit 20.

Meanwhile, in the measurement mode represented by a period from time t₅ to time t₆, the biometric information measuring apparatus 10 sets the light quantity emitted from the light emitting element 1A as the light quantity Q₂ until the emission of the light emitting element 1A is stopped at the step S110 of FIG. 9.

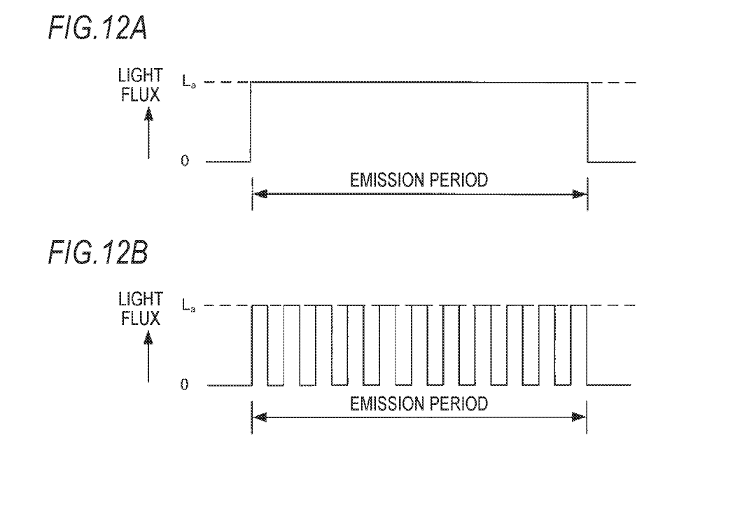

As used herein, the emission of the light emitting element 1A is intended to include not only a case where light is continuously emitted with a predetermined light flux (for example, the light flux Lₐ) over the entire emission period, as illustrated in FIG. 12A, but also a state in which the emission and the stop of emission of light are repeated with a predetermined light flux (for example, the light flux Lₐ), as illustrated in FIG. 12B. Since the upper limit frequency of the spectral distribution 82 of the light reflected by the blood vessel 6 is about 20 kHz, when the emission of light and the stop of emission of light are repeated in the emission period, the spectral distribution 82 may be obtained when the light emitting element 1A emits light at twice the upper limit frequency, that is, about 40 kHz.

FIG. 11 illustrates an example of controlling the length of the emission period of the light emitting element 1A so as to control the magnitude of the light quantity emitted from the light emitting element 1A so that the light quantity in the measurement mode becomes larger than the light quantity in the standby mode. However, the biometric information measuring apparatus 10 may control the magnitude of the light quantity emitted from the light emitting element 1A, for example by changing the light flux emitted from the light emitting element 1A.

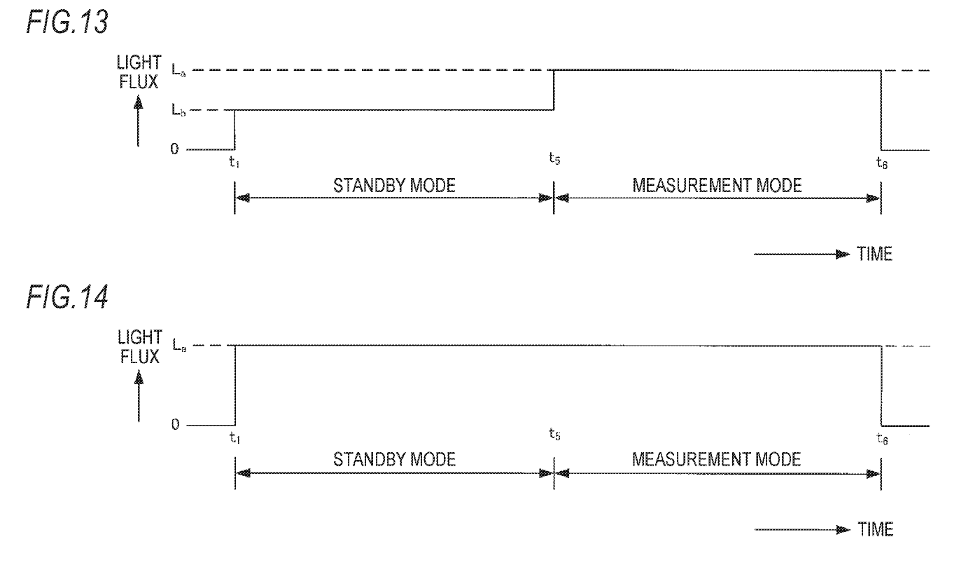

For example, as illustrated in FIG. 13, the biometric information measuring apparatus 10 may cause the light emitting element 1A to emit light with a light flux L_{b} smaller than the light flux Lₐ in the standby mode and to emit light with the light flux Lₐ in the measurement mode.

Further, the biometric information measuring apparatus 10 may control the magnitude of the light quantity emitted from the light emitting element 1A by changing the emission period of the light emitting element 1A and the light flux emitted from the light emitting element 1A.

In a situation where the biometric information measuring apparatus 10 is incorporated in a smartphone, when the display device 34 such as a display on which the measurement start button is displayed is located on a front surface and the light emitting element 1A and the light receiving element 3 are located on a rear surface, the user presses the measurement start button, turns over the smartphone, and places the living body 8 such as a finger at the measurement position.

At this time, since the light quantity Q₁ in the standby mode after the measurement start button is pressed is smaller than the light quantity Q₂ in the measurement mode in which the living body 8 is detected to start the measurement of the blood flow rate, the light quantity emitted from the light emitting element 1A toward the body of the user unintentionally may be reduced when the user turns over the smartphone in an attempt to place his/her finger at the measurement position as compared with the case where the light quantity Q₁ used in the standby mode becomes equal to the light quantity Q₂ used in the measurement mode.

In addition, since the light quantity emitted from the light emitting element 1A is limited to fall within a range that does not affect the user's body, there is no particular problem even when the user's body is irradiated with the light of the light emitting element 1A, but it may be considered that there are some users who feel a stress when the user's body is irradiated with the light.

Therefore, by making the light quantity in the standby mode smaller than the light quantity in the measurement mode to reduce the light quantity with which the user's body may be unintentionally irradiated, the user's stress caused by the light irradiation on the body is relaxed. Further, irrespective of whether or not light is unintentionally emitted from the light emitting element 1A toward the user's body, by setting the light quantity in the standby mode to be smaller than the light quantity in the measurement mode, the power consumption in the standby mode is reduced as compared with a case where the light quantity in the standby mode is not decreased.

In addition, in the biometric information measuring process illustrated in FIG. 9, when the living body 8 has not been detected at the step S80, the emission of the light emitting element 1A is stopped to stop the measurement of the blood flow rate. However, the process after the living body 8 is not detected at the step S80 is not limited thereto.

For example, after notifying the user that the living body 8 has been separated from the measurement position at the step S90, the process may proceed to the step S20 to return to the standby mode again. In this case, when the living body 8 is placed at the measurement position, the blood flow rate is measured again after shifting to the measurement mode. Therefore, even when the user's body unintentionally moves and the living body 8 is temporarily separated from the measurement position, the blood flow rate is measured again without the user's pressing the measurement start button.

At this time, in order to notify the user whether the biometric information measuring apparatus 10 is in the standby mode or the measurement mode, the biometric information measuring apparatus 10 may change the contents displayed on the display device 34 for each mode.

For example, the biometric information measuring apparatus 10 does not display anything on the display device 34 in the standby mode. When a shift to the measurement mode is made in which the measurement of the blood flow rate is started, the biometric information measuring apparatus 10 causes the display device 34 to display a notification of the measurement start to the user and the measurement result of the blood flow rate using a displaying method such as numerical values, graphs, characters or the like through which the user can recognize the measurement result.

In addition, the biometric information measuring apparatus 10 may stop the supply of power to the display device 34 in the standby mode and may resume the supply of power to the display device 34 when shifting to the measurement mode so that information is displayed on the display device 34. Also in this case, since some information is displayed on the display device 34 when the biometric information measuring apparatus 10 shifts to the measurement mode, the user may check whether the biometric information measuring apparatus 10 is in the standby mode or the measurement mode. Furthermore, the power consumption in the biometric information measuring apparatus 10 may be suppressed as compared with a case where power is constantly supplied to a device or the like included in the biometric information measuring apparatus 10 regardless of a mode.

As used herein, the phrase “stopping the measurement” in the biometric information measuring apparatus 10 refers to that the biometric information measuring apparatus 10 shifts from the measurement mode to another mode (another state) such as the standby mode. For example, the phrase “stopping the measurement” includes not only performing no measurement of the biometric information but also setting the contents to be displayed on the display device 34 as described above or the supply state of power in the biometric information measuring apparatus 10 to be different from the measurement mode although the measurement of the biometric information itself is continued.

In this way, the biometric information measuring apparatus 10 according to the first exemplary embodiment uses the spectral distribution 82 of the light reflected by the living body 8 or the light transmitted through the living body 8 to shift from the standby mode to the measurement mode upon detecting that the living body 8 is placed at the measurement position of the biometric information measuring apparatus 10. Therefore, the operability at the time of starting the measurement of the biometric information is improved as compared with a case where the measurement of the biometric information is started by pressing a button or the like after the living body 8 is placed at the measurement position of the biometric information measuring apparatus 10.

Meanwhile, the functional units included in the biometric information measuring apparatus 10 according to the first exemplary embodiment may be distributed to other devices and may be interconnected by a network so as to constitute the biometric information measuring apparatus 10. For example, the measuring unit 22 may be arranged in another device on the network and the biometric information measuring apparatus 10 may transmit the spectral distribution 82 detected by the detecting unit 20 to the measuring unit 22 arranged in another device via the communication device 37 and may receive a measurement result of the biometric information measured by the measuring unit 22 and notify the received measurement result to the user.

### First Modification Example of First Exemplary Embodiment

In the above-described biometric information measuring apparatus 10, the light quantity Q₁ in the standby mode is set to be smaller than the light quantity Q₂ in the measurement mode, but the light quantity Q₂ in the measurement mode is limited to fall within a range that does not affect the user's body. Therefore, as illustrated in FIG. 14, the biometric information measuring apparatus 10 may drive the light emitting element 1A such that both of the light fluxes in the standby mode and the measurement mode are set to the light flux Lₐ, and the light quantity per unit time in the standby mode becomes equal to that in the measurement mode.

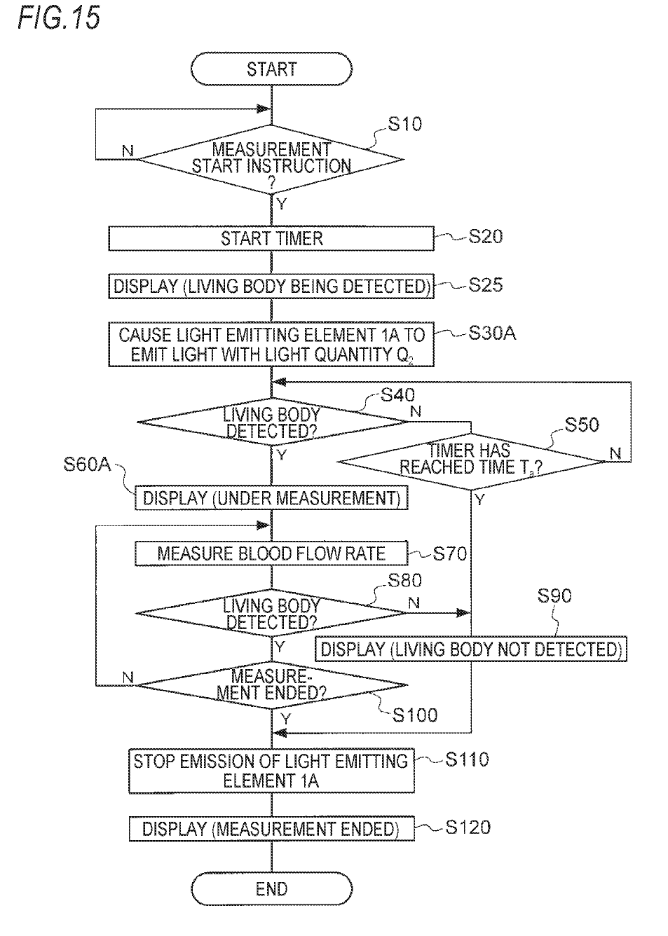

FIG. 15 is a flowchart illustrating an example of a flow of a biometric information measuring process executed by the CPU 30 when a smartphone in which the biometric information measuring apparatus 10 is incorporated is powered on.

The biometric information measuring process illustrated in FIG. 15 is different from the biometric information measuring process illustrated in FIG. 9 in that the step S30 is replaced with the step S30A, the step S60 is replaced with the step S60A and steps S25 and S120 are newly added.

At the step S25, the CPU displays a message such as “the living body is being detected” on, for example, the display device 34 to instruct the user to place the living body 8 such as a finger or the like at the measurement position.

At the step S30A, the CPU 30 controls the driving circuit 14 such that the light emitting element 1A emits light with the same light quantity Q₂ as in the measurement mode.

Then, when the living body 8 is detected at the step S40, at the step S60A, the CPU 30 notifies the user of information indicating that the blood flow rate is being measured. For example, a message such as “The blood flow rate is being measured” or the like is displayed on the display device 34 and the user is notified of the fact that the biometric information is being measured.

When a measurement end instruction is received in the measurement mode or when the living body 8 is no longer detected, at the step S120, the CPU 30 notifies the user of information indicating the measurement end. For example, a message such as “The measurement of blood flow rate is finished” or the like is displayed on the display device 34 to notify the user that the measurement of biometric information is stopped. In addition, when a predetermined time has elapsed or acquisition of information required for the measurement is completed, the information indicating the end of measurement may be notified to the user.

In this manner, according to the biometric information measuring process illustrated in FIG. 15, the light quantity per unit time in each of the standby mode and the measurement mode is set to the light quantity Q₂. In the first modification example, the steps S30A and S40 are preparatory states of measuring biometric information for transition to the measurement mode and correspond to the standby mode.

In the first modification example, since there is no change in light quantity for each mode emitted from the light emitting element 1A, it is difficult for the user to determine whether the biometric information measuring apparatus 10 is in the standby mode or in the measurement mode from the emission state of the light emitting element 1A. Therefore, although the process of notifying to the user at the steps S25, S60A and S120 in FIG. 15 is not necessary, since the operation state of the biometric information measuring apparatus 10 is notified to the user in each of these steps, the user may receive a sense of security that the biometric information measuring apparatus 10 is operating normally.

The method of notifying information to the user at the steps S25, S60A and S120 is not limited to the displaying on the display device 34. For example, the user may be notified of the information by outputting a voice from the speaker 36 or vibrating the vibration element 33.

### Second Modification Example of First Exemplary Embodiment

The exemplary embodiment in which the light emitting element 1A emits light based on the measurement start instruction from the user has been described in the first exemplary embodiment. In the second modification example, an exemplary embodiment in which the light emitting element 1A emits light based on both of the measurement start instruction from the user and the state of external light will be described.

Here, since the biometric information measuring apparatus 10 is configured to perform the measurement in a state of being in contact with the living body, that is, in a state in which external light hardly enters the light receiving element 3, the quantity of light received by the light receiving element 3 is very small in a state where the emission of the light emitting element 1A is stopped. Therefore, when the quantity of light received by the light receiving element 3 is large in the state where the emission of the light emitting element 1A is stopped, it may be determined that no contact with the living body is made.

Meanwhile, when an attempt is made to detect the presence or absence of a living body only with the quantity of light received by the light receiving element 3 in the state where the emission of the light emitting element 1A is stopped, for example, when an illumination of the room is turned OFF in a state where an object other than the living body 8 is placed at the measurement position or in a state that nothing is placed at the measurement position, erroneous detection may be made that the living body 8 is placed.

Therefore, in the second modification example, when there is a measurement start instruction from the user and the quantity of light received by the light receiving element 3 in the state where the emission of the light emitting element 1A is stopped is smaller than a predetermined received light quantity, it is determined that the living body is likely to be placed, and preliminary emission is made for transition to the measurement mode. Then, when the spectral intensity detected by the preliminary emission is actually the intensity indicating the living body, the biometric information measuring apparatus 10 shifts to the measurement mode. That is, even when there is a measurement start instruction from the user, the light emitting element 1A does not perform the preliminary emission for transition to the measurement mode when the quantity of light received by the light receiving element 3 is large in the state where the emission of the light emitting element 1A is stopped.

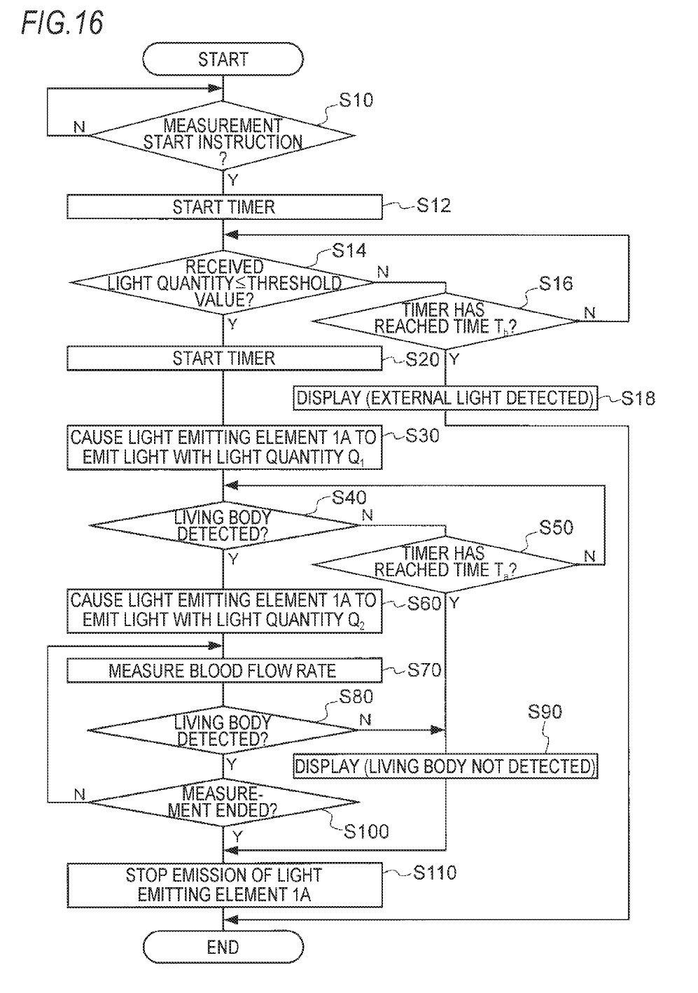

FIG. 16 is a flowchart illustrating an example of a flow of the biometric information measuring process executed by the CPU 30 when a smartphone in which the biometric information measuring apparatus 10 is incorporated is powered on.

The biometric information measuring process illustrated in FIG. 16 is different from the biometric information measuring process illustrated in FIG. 9 in that steps S12 to S18 are added.

After receiving the measurement start instruction at step S10, at the step S12, the CPU 30 starts the timer incorporated in the CPU 30.

At the step S14, the CPU 30 calculates the quantity of received light per unit time, for example, by using the intensity of light received by the light receiving element 3 and digitized by the A/D converting circuit 18 in a state where the light emitting element 1A emits no light. Then, the CPU 30 determines whether or not the calculated received light quantity is equal to or smaller than a predetermined received light quantity (received light quantity threshold value). In this case, the received light quantity threshold value may be set to a received light quantity at or below which the living body is thought to be placed. The specific value of the received light quantity threshold value is determined by experiments performed by the biometric information measuring apparatus 10 as an actual apparatus or a computer simulation based on the design specifications of the biometric information measuring apparatus 10.

When the determination at the step S14 is negative, that is, when the quantity of received light by external light exceeds the received light quantity threshold value, it is determined that no living body is placed, and the process proceeds to the step S16.

At the step S16, the CPU 30 determines whether or not the elapsed time of the timer started at the step S12 has become equal to or longer than time T_{b}. Time T_{b} is a value defining a period in which the quantity of received light by external light is compared to the received light quantity threshold value at the step S14. When the elapsed time of the timer is less than time T_{b}, the process proceeds to the step S14 and repeats the processes through the steps S14 and S16 until the quantity of received light by external light becomes equal to or less than the received light quantity threshold value.

When the elapsed time of the timer is equal to or more than time T_{b}, the process proceeds to the step S18 where the CPU 30 displays a message such as “The measurement is stopped” or the like on, for example, the display device 34, and then the biometric information measuring process is ended.

Meanwhile, when the determination at the step S14 is affirmative, that is, when the quantity of received light by external light is equal to or less than the received light quantity threshold value, it is determined that the living body is likely to be placed, and then the process proceeds to the step S20.

Thereafter, the CPU 30 measures the biometric information by performing the processes through the steps S20 to S110 previously described in FIG. 9.

In this way, the biometric information measuring apparatus 10 according to the second modification example shifts from the standby mode to the measurement mode to measure the biometric information when the quantity of received light by external light is equal to or less than the received light quantity threshold value and the living body 8 is detected at the measurement position.

Therefore, it is unnecessary to cause the light emitting element 1A to emit light in a preparatory manner from when there is a measurement start instruction from the user to when the living body is actually placed. Further, as compared with the case of shifting from the standby mode to the measurement mode to measure the biometric information, when the quantity of received light by external light is equal to or less than the received light quantity threshold value without checking whether or not the living body 8 is placed at the measurement position, it is possible to suppress the emission as the measurement mode despite the placement of an object other than the living body.

In the flowchart illustrated in FIG. 16, the light emitting element 1A is not caused to emit light until the quantity of received light by external light becomes equal to or less than the received light quantity threshold value. However, the emission pattern of the light emitting element 1A is not limited thereto.

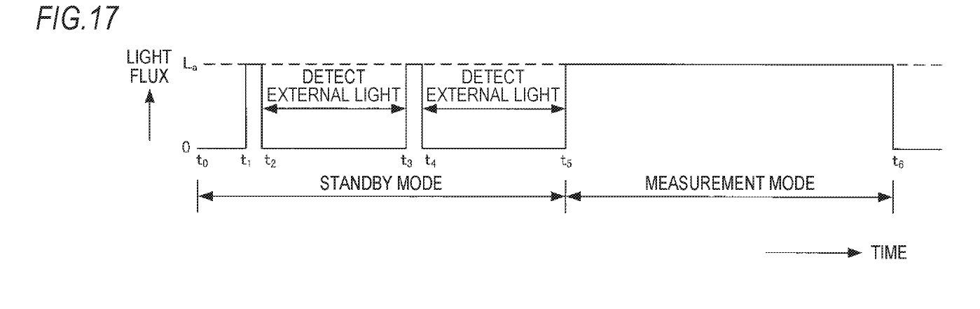

For example, as illustrated in FIG. 17, the light emitting element 1A may be caused to emit light in such a manner that an emission period and a non-emission period alternately appear in the standby mode. In this case, the biometric information measuring apparatus 10 may detect whether or not the living body 8 is placed at the measurement position during the emission period (t₁ to t₂ and t₃ to t₄) during which the light emitting element 1A is emitting light, and may determine whether or not the quantity of received light by external light is equal to or less than the received light quantity threshold value during the non-emission period (t₂ to t₃ and t₄ to t₅) during which the light emitting element 1A is emitting no light.

### Second Exemplary Embodiment

In the first exemplary embodiment, the biometric information measuring apparatus 10 that measures the blood flow rate as an example of the biometric information has been described. In a second exemplary embodiment, a biometric information measuring apparatus 10A that measures plural pieces of biometric information in the measurement mode will be described. Specifically, the biometric information measuring apparatus 10A that measures a blood flow rate of the blood in the blood vessel 6 and an oxygen saturation in the blood in the measurement mode will be described.

Similarly to the biometric information measuring apparatus 10 according to the first exemplary embodiment, the biometric information measuring apparatus 10A according to the second exemplary embodiment is capable of measuring the blood flow rate and the blood oxygen saturation on the same principle using either the light transmitted through the blood vessel 6 of the living body 8 or the light reflected by the blood vessel 6 of the living body 8. Therefore, as an example, a case where the blood flow rate and the blood oxygen saturation are measured using the light reflected by the blood vessel 6 of the living body 8 will be described below.

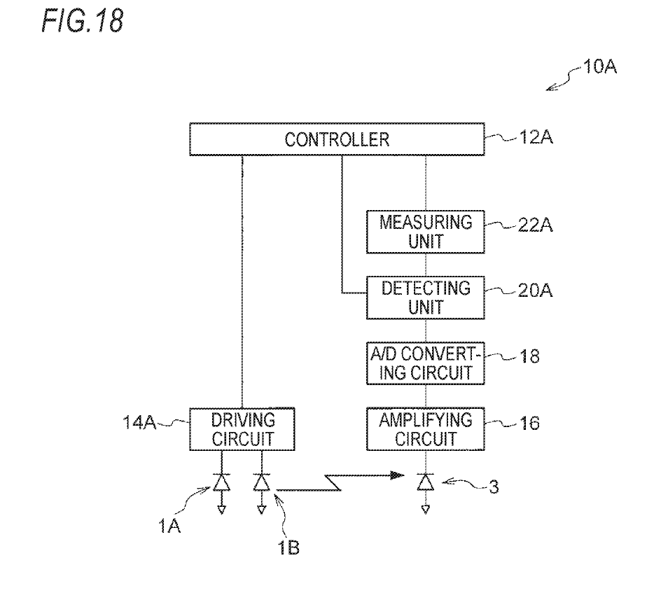

FIG. 18 illustrates a configuration example of the biometric information measuring apparatus 10A according to the second exemplary embodiment. The configuration example of the biometric information measuring apparatus 10A illustrated in FIG. 18 is different from that of the biometric information measuring apparatus 10 according to the first exemplary embodiment illustrated in FIG. 1 in that a light emitting element 1B is added. With the addition of the light emitting element 1B, a controller 12A, a driving circuit 14A, a detecting unit 20A and a measuring unit 22A perform processes different from the controller 12, the driving circuit 14, the detecting unit 20 and the measuring unit 22 illustrated in FIG. 1, respectively. Hereinafter, parts different from those of the biometric information measuring apparatus 10 according to the first exemplary embodiment will be described.

The light emitting element 1B is an element that emits a laser beam, like the light emitting element 1A as an example. In this case, the light emitting element 1B may be either a surface emitting laser element or an edge emitting laser element, but an element that emits light having a wavelength different from that of the light emitting element 1A is used. As an example, it is assumed that the light emitting element 1A emits light having an infrared (IR) wavelength and the light emitting element 1B emits light having a red light wavelength. Note that the light emitting element 1B is not limited to a laser element that emits a laser beam but may be an LED element such as a light emitting diode (LED) or an organic light emitting diode (OLED).

According to an instruction from the controller 12A to be described later, the driving circuit 14A supplies, for example, power for driving each of the light emitting element 1A and the light emitting element 1B, to drive the light emitting element 1A and the light emitting element 1B so that the light emitting element 1A and the light emitting element 1B individually emit light or stop the emission.

The detecting unit 20A performs FFT processing on the temporal change in the light intensity digitized by the A/D converting circuit 18, detects the spectral distribution for each frequency ω, and outputs the detected spectral distribution and the time-series light intensities to the measuring unit 22A.

The controller 12A receives various instructions from the user and determines from the spectral distribution detected by the detecting unit 20A whether or not the light reflected by the blood vessel 6 of the living body 8 has been received by the light receiving element 3. When it is determined that the light reflected by the blood vessel 6 of the living body 8 has been received by the light receiving element 3, the controller 12A shifts the operation state of the biometric information measuring apparatus 10A from the standby mode (standby state) to the measurement mode (measurement state). As an example, based on the spectral distribution 82 detected by the detecting unit 20A and the intensity of light received by the light receiving element 3, the controller 12A controls the driving circuit 14A and the measuring unit 22A to start measurement of the blood flow rate and the blood oxygen saturation, and shifts the biometric information measuring apparatus 10A from the standby mode to the measurement mode.

Meanwhile, upon receiving a measurement end instruction from the user in a state where the biometric information measuring apparatus 10A is already in the measurement mode, the controller 12A controls the driving circuit 14A and the measuring unit 22A to stop the measurement of the blood flow rate and the blood oxygen saturation.

In addition, when the living body 8 is no longer detected in the state where the biometric information measuring apparatus 10A is already in the measurement mode, without receiving the measurement end instruction from the user, the controller 12A controls the driving circuit 14A and the measuring unit 22A to stop the measurement of the blood flow rate and the blood oxygen saturation.

According to an instruction of the controller 12A, the measuring unit 22A measures the blood flow rate and the blood oxygen saturation based on the spectral distribution 82 detected by the detecting unit 20A and the intensity of light received by the light receiving element 3.

Next, the principle of measurement of the blood oxygen saturation in the biometric information measuring apparatus 10A will be described.

A blood oxygen saturation is an index indicating how much hemoglobin in the blood is combined with oxygen. Symptoms such as anemia are likely to occur as the blood oxygen saturation decreases.

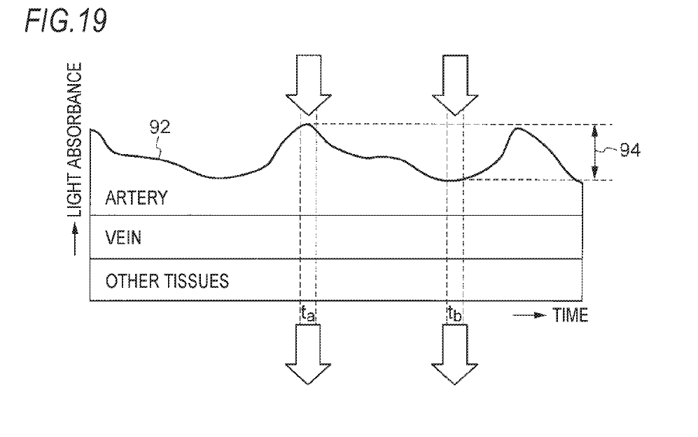

FIG. 19 is a conceptual view illustrating a change in quantity (absorbance) of light absorbed in the living body 8, for example. As illustrated in FIG. 19, the light absorbance in the living body 8 tends to be varied with the lapse of time.

Further, from the breakdown related to the variation of the light absorbance in the living body 8, it is known that the light absorbance is mainly varied by an artery while a variation in other tissues including veins and stationary tissues is small, and thus may be considered non-variable in the light absorbance compared to the artery. This is because arterial blood pumped from the heart moves through a blood vessel with a pulse wave and accordingly the artery is stretched and contracted with time along the cross-sectional direction of the artery to change the thickness of the artery. In FIG. 19, a range indicated by an arrow 94 represents a variation in the light absorbance corresponding to the change in the thickness of the artery.

In FIG. 19, when the light intensity at time tₐ is denoted by Iₐ and the light intensity at time t_{b} is denoted by I_{b}, a variation ΔA in the light absorbance by the change in the thickness of the artery is expressed by the following equation (1).

ΔA=ln(I_{b}/Iₐ) (1)

Incidentally, it is known that hemoglobin (oxygenated hemoglobin) combined with oxygen flowing through an artery easily absorbs light in an infrared (IR) region having a wavelength in the vicinity of about 880 nm and hemoglobin (reduced hemoglobin) not combined with oxygen easily absorbs light in a red region having a wavelength in the vicinity of about 665 nm. Further, it is known that the blood oxygen saturation is proportional to the ratio of the variation ΔA of the light absorbance at different wavelengths.

Therefore, by using infrared light (IR light) and red light which are likely to cause a difference in light absorbance between oxygenated hemoglobin and reduced hemoglobin as compared with combinations of other wavelengths, the ratio of variation ΔA_{IR} of the light absorbance when the living body 8 is irradiated with the IR light to variation ΔA_{Red} of the light absorbance when the living body 8 is irradiated with the red light is calculated so as to calculate the blood oxygen saturation S according to the following equation (2). In the equation (2), k is a proportional constant.

S=k(ΔA_{Red}/ΔA_{IR}) (2)

That is, when the blood oxygen saturation is calculated, the light emitting elements 1A and 1B that emit lights of different wavelengths respectively, specifically, the light emitting element 1A that emits the IR light and the light emitting element 1B that emits the red light, may perform the emission so that their respective emission periods do not overlap with each other although the emission periods may partially overlap with each other. Then, the reflected light from each of the light emitting elements 1A and 1B is received by the light receiving element 3 and the blood oxygen saturation is measured by calculating the equation (1) and equation (2) from the light intensity at each point of time of reception of the reflected light or a known equation, which is obtained by modifying the equation (1) and equation (2), from the light intensity at each point of time of reception of the reflected light.

As the known equation obtained by modifying the above equation (1), for example, the equation (1) may be deployed to express the variation ΔA in the light absorbance as the following equation (3).

ΔA=ln I_{b}−ln Iₐ (3)

Alternatively, the equation (1) may be modified into the following equation (4).

ΔA=ln(I_{b}/Iₐ)=ln(1+(I_{b}−Iₐ)/Iₐ) (4)

Typically, since (I_{b}−Iₐ)<<Iₐ, the relationship of ln(I_{b}/Iₐ)≈(I_{b}−Iₐ)/Iₐ is established. Therefore, instead of the equation (1), the following equation (5) may be used as the variation ΔA in the light absorbance.

ΔA≈(I_{b}−Iₐ)/Iₐ (5)

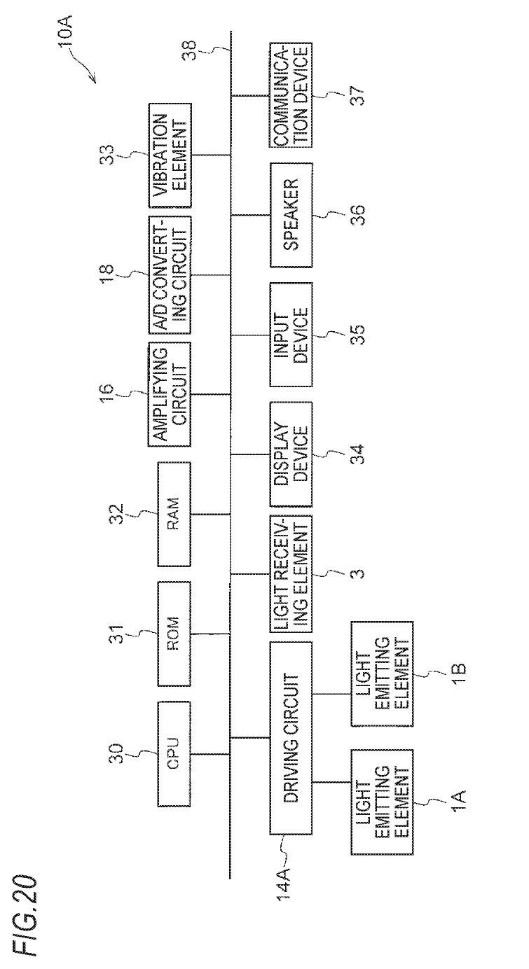

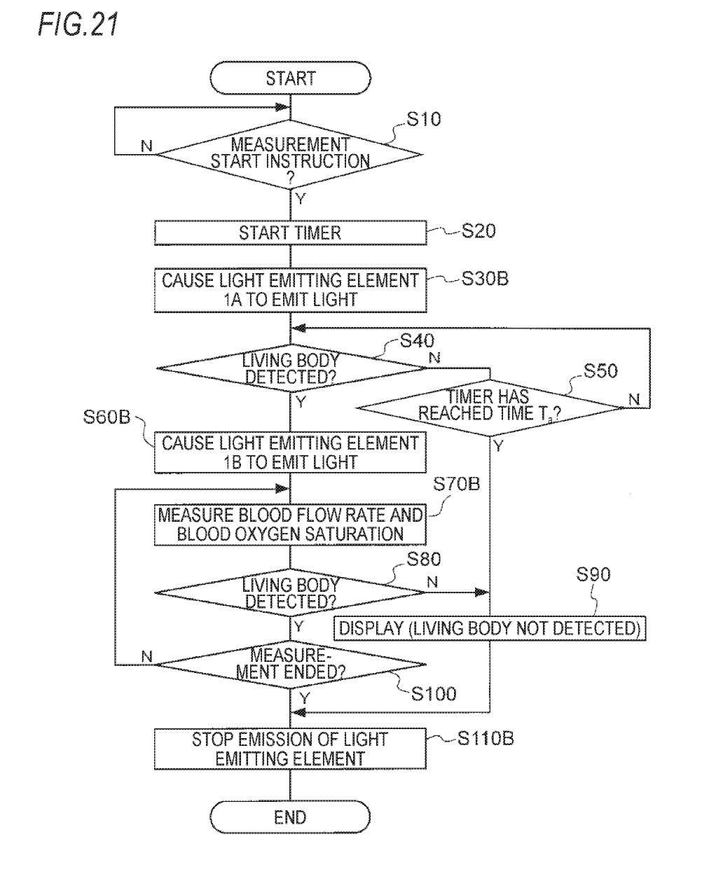

Next, a configuration of a main part of an electric system of the biometric information measuring apparatus 10A according to the second exemplary embodiment will be described with reference to FIG. 20. Similarly to the biometric information measuring apparatus 10 according to the first exemplary embodiment, descriptions will be given with the presumption that the biometric information measuring apparatus 10A is incorporated in a portable terminal such as a smartphone.

The configuration of a main part of an electrical system of the biometric information measuring apparatus 10A illustrated in FIG. 20 is different from that of the electrical system of the biometric information measuring apparatus 10 according to the first exemplary embodiment illustrated in FIG. 8 in that a light emitting element 1B is newly added and accordingly the driving circuit 14 that drives the light emitting element 1A is replaced with a driving circuit 14A for driving the light emitting element 1A and the light emitting element 1B. Other configurations are the same as those of the biometric information measuring apparatus 10.

Next, the operation of the biometric information measuring apparatus 10A will be described. FIG. 21 is a flowchart illustrating an example of a flow of a biometric information measuring process executed by the CPU 30 when a smartphone in which the biometric information measuring apparatus 10A is incorporated is powered on.

The biometric information measuring process illustrated in FIG. 21 is different from the biometric information measuring process according to the first exemplary embodiment illustrated in FIG. 9 in that the steps S30, S60, S70 and S110 are replaced with steps S30B, S60B, S70B and S110B, respectively. Other processes are the same as those of the biometric information measuring process according to the first exemplary embodiment.

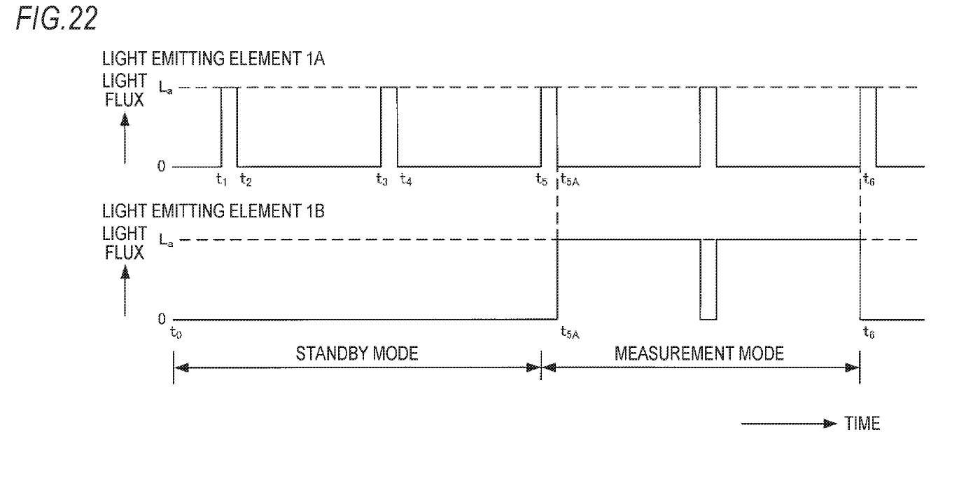

Upon receiving a measurement start instruction from the user, at the step S30B, the CPU 30 controls the driving circuit 14A to cause the light emitting element 1A to emit light with a predetermined light quantity, as illustrated in FIG. 22. In the example of FIG. 22, the CPU 30 drives the light emitting element 1A such that an emission period and a non-emission period appear alternately. A duty ratio set when the light emitting element 1A is driven is not particularly limited and an emission period of the light emitting element 1A may be set to such a length that the living body 8 may be detected and a blood flow rate may be measured.

In this way, in the standby mode, the light emitting element 1A is caused to emit light while the light emitting element 1B is prevented from emitting light.

Then, when the living body 8 is detected at the measurement position based on the spectral distribution of light received by the light receiving element 3 at the step S40 and the biometric information measuring apparatus 10A shifts from the standby mode to the measurement mode, at the step S60B, the CPU 30 controls the driving circuit 14A to cause the light emitting element 1B to emit light with a predetermined light quantity, as illustrated in FIG. 22.

In this case, the CPU 30 may control the driving circuit 14A to cause the light emitting element 1B to emit light during the non-emission period of the light emitting element 1A. This is because, when the emission period of the light emitting element 1A and the emission period of the light emitting element 1B overlap with each other, lights emitted therefrom may interfere with each other, whereby an error is likely to be included in results of measurement of the blood flow rate and the blood oxygen saturation.

In the step S70B, the CPU 30 uses the spectral distribution 82 of light received by the light receiving element 3 in the emission period of the light emitting element 1A to calculate a blood volume and a blood velocity in accordance with the method described in the first exemplary embodiment, measures a blood flow rate from the product of the blood volume and the blood velocity, and stores the measurement result in the RAM 32, for example.

In addition, the CPU 30 uses the intensity of light received by the light receiving element 3 during the emission period of the light emitting element 1A and the intensity of light received by the light receiving element 3 during the emission period of the light emitting element 1B to measure a blood oxygen saturation according to the above-described equations (1) and (2), and stores a result of the measurement in the RAM 32, for example.

Then, in the measurement mode, when a measurement end instruction is received from the user or the living body 8 is no longer detected at the measurement position, at the step S110B, the CPU 30 controls the driving circuit 14A to stop the emission of the light emitting element 1A and the light emitting element 1B so as to stop the measurement of the blood flow rate and the blood oxygen saturation.

In this way, with the biometric information measuring apparatus 10A according to the second exemplary embodiment, when the spectral distribution 82 of light of the light emitting element 1A reflected by or transmitted through the living body 8 is used to detect that the living body 8 is placed at the measurement position of the biometric information measuring apparatus 10A, the light emitting element 1B emits light to start measurement of plural pieces of biometric information such as, for example, the blood flow rate and the blood oxygen saturation. Therefore, the operability at the start of measurement of the biometric information is improved as compared with a case where the living body 8 is placed at the measurement position of the biometric information measuring apparatus 10A and then measurement of the biometric information is started by pressing a button or the like.

Further, the biometric information measuring apparatus 10A allows only the light emitting element 1A to emit light in the standby mode to detect the living body 8, and allows both of the light emitting element 1A and the light emitting element 1B to emit light after shifting to the measurement mode. Therefore, the light quantity emitted toward the body of the user unintentionally may be reduced when the user turns over the smartphone in an attempt to place the living body 8 such as his/her finger or the like at the measurement position as compared with a case where the light emitting element 1A and the light emitting element 1B are caused to emit light in the standby mode.

The contents and various modification examples suggested to the biometric information measuring apparatus 10 according to the first exemplary embodiment are also applied to the biometric information measuring apparatus 10A.

For example, after the user is notified that the living body 8 is separated from the measurement position in the step S90, the emission of the light emitting element 1B may be stopped and the process may proceed to the step S20 to return again to the standby mode. In this case, since the light emitting element 1A continues to emit light with the light quantity Q₁, the biometric information measuring apparatus 10A detects the living body 8 without receiving a measurement start instruction again from the user, and shifts to the measurement mode when the living body 8 is detected.

Further, in order to notify the user whether the biometric information measuring apparatus 10A is in the standby mode or in the measurement mode, the biometric information measuring apparatus 10A may change the contents displayed on the display device 34 for each mode.

Further, the biometric information measuring apparatus 10A may stop the supply of power to the display device 34 in the standby mode and may start the supply of power to the display device 34 at the time of shifting to the measurement mode to display information on the display device 34.

Furthermore, as described in the first modification example of the first exemplary embodiment, the biometric information measuring apparatus 10A may allow the light emitting element 1B to emit light in the non-emission period of the light emitting element 1A even in the standby mode to make the light quantity in the standby mode equal to the light quantity in the measurement mode. In this case, even when the biometric information measuring apparatus 10A is in the standby mode, although there is a case that the same light quantity as in the measurement mode is emitted toward the user's body, there is no particular problem because the light quantity in the measurement mode is limited to fall within a range that does not affect the user's body, as described previously.

Moreover, as described in the second modification example of the first exemplary embodiment, when the quantity of received light by external light is equal to or less than a received light quantity threshold value and the living body 8 is detected at the measurement position, the biometric information measuring apparatus 10A may shift from the standby mode to the measurement mode to measure the biometric information.

In the biometric information measuring apparatus 10A, as an example, one light emitting element 1A and one light emitting element 1B are included. However, for example, two or more light emitting elements 1A and two or more light emitting elements 1B may be included.

### Third Exemplary Embodiment

In the first exemplary embodiment, the light emitting element 1A is used for both of the case of detecting the living body 8 and the case of measuring the blood flow rate. A third exemplary embodiment employs a biometric information measuring apparatus 10B including a light emitting element for detecting the living body 8 and a light emitting element for measuring biometric information individually, as will be described below.

In addition, similarly to the biometric information measuring apparatus 10 according to the first exemplary embodiment, the biometric information measuring apparatus 10B according to the third exemplary embodiment is used to measure the biometric information such as, for example, a blood flow rate, as will be described below. In this case, it may be possible to measure the blood flow rate with the same principle using either the light transmitted through the blood vessel 6 of the living body 8 or the light reflected by the blood vessel 6 of the living body 8. However, hereinafter, as an example, a case where the blood flow rate is measured using the light reflected by the blood vessel 6 of the living body 8 will be described.

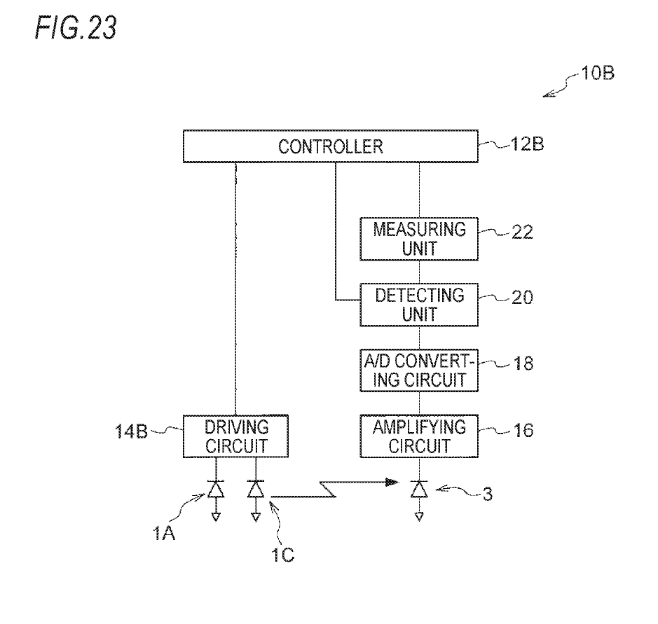

FIG. 23 illustrates a configuration example of the biometric information measuring apparatus 10B according to the third exemplary embodiment. The configuration example of the biometric information measuring apparatus 10B illustrated in FIG. 23 is different from the configuration example of the biometric information measuring apparatus 10 according to the first exemplary embodiment illustrated in FIG. 1 in that a light emitting element 1C is added. With the addition of the light emitting element 1C, a controller 12B and a driving circuit 14B perform processes different from the controller and the driving circuit 14 illustrated in FIG. 1, respectively. Hereinafter, parts different from those of the biometric information measuring apparatus 10 according to the first exemplary embodiment will be described.

The light emitting element 1C is an element that emits a laser beam, like the light emitting element 1A. In this case, the light emitting element 1C may be either a surface emitting laser element or an edge emitting laser element, but an element that emits light having a wavelength different from that of the light emitting element 1A is used. Specifically, the light emitting element 1C may have such a wavelength to more clearly exhibit a difference between the spectral distribution 82 of light of the light emitting element 1C reflected by the living body 8 and the spectral distribution 83 by external light.

According to an instruction from the controller 12B to be described later, the driving circuit 14B supplies, for example, power for driving each of the light emitting element 1A and the light emitting element 1C, to drive the light emitting element 1A and the light emitting element 1C so that the light emitting element 1A and the light emitting element 1C individually emit light or stop the emission.

The controller 12B receives various instructions from the user, causes the light emitting element 1C to emit light with the light quantity Q₁ in the standby mode, and determines from the spectral distribution detected by the detecting unit 20 whether or not the light reflected by the blood vessel 6 of the living body 8 has been received by the light receiving element 3. When it is determined that the light reflected by the blood vessel 6 of the living body 8 has been received by the light receiving element 3, the controller 12B controls the driving circuit 14B and the measuring unit 22 to start measurement of a blood flow rate, based on the spectral distribution 82 detected by the detecting unit 20, and shifts the biometric information measuring apparatus 10B from the standby mode to the measurement mode.

Meanwhile, upon receiving a measurement end instruction from the user in a state where the biometric information measuring apparatus 10B is already in the measurement mode, the controller 12B controls the driving circuit 14B and the measuring unit 22 to stop the measurement of the blood flow rate.

In addition, when the living body 8 is no longer detected in the state where the biometric information measuring apparatus 10B is already in the measurement mode, without receiving the measurement end instruction from the user, the controller 12B controls the driving circuit 14B and the measuring unit 22 to stop the measurement of the blood flow rate.

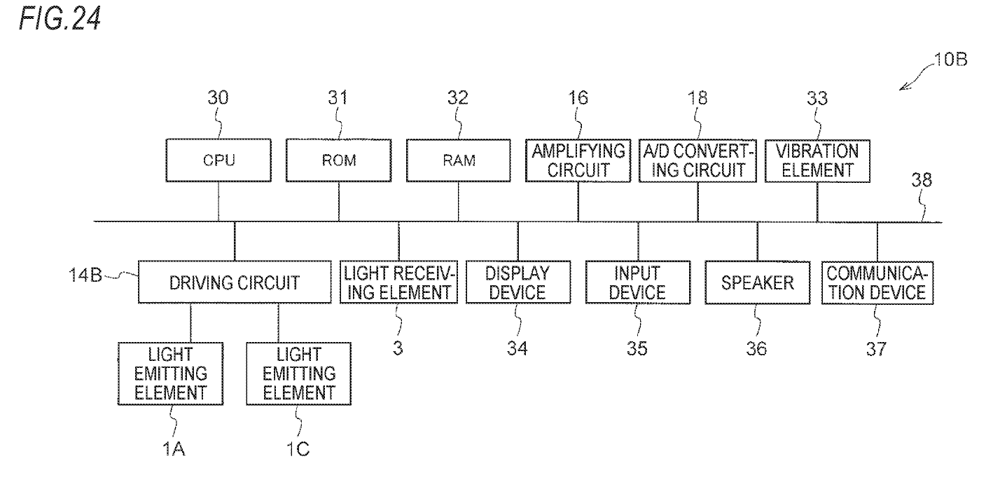

Next, a configuration of a main part of an electric system of the biometric information measuring apparatus 10B according to the third exemplary embodiment will be described with reference to FIG. 24. Similarly to the biometric information measuring apparatus 10 according to the first exemplary embodiment, descriptions will be given with the presumption that the biometric information measuring apparatus 10B is incorporated in a portable terminal such as a smartphone.

The configuration of a main part of an electrical system of the biometric information measuring apparatus 10B illustrated in FIG. 24 is different from that of the electrical system of the biometric information measuring apparatus 10 according to the first exemplary embodiment illustrated in FIG. 8 in that the light emitting element 1C is newly added and accordingly the driving circuit 14 that drives the light emitting element 1A is replaced with the driving circuit 14B for driving the light emitting element 1A and the light emitting element 1C. Other configurations are the same as those of the biometric information measuring apparatus 10.

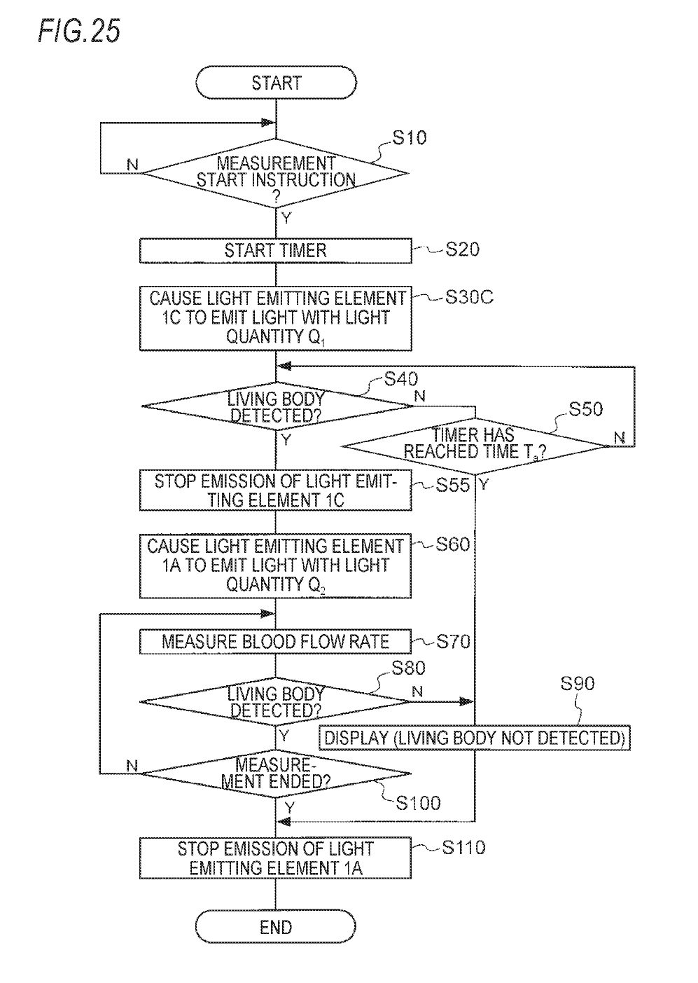

Next, the operation of the biometric information measuring apparatus 10B will be described. FIG. 25 is a flowchart illustrating an example of a flow of a biometric information measuring process executed by the CPU 30 when a smartphone in which the biometric information measuring apparatus 10B is incorporated is powered on.

The biometric information measuring process illustrated in FIG. 25 is different from the biometric information measuring process according to the first exemplary embodiment illustrated in FIG. 9 in that the step S30 is replaced with the step S30C and step S55 is newly added. Other processes are the same as those of the biometric information measuring process according to the first exemplary embodiment.

Upon receiving a measurement start instruction from the user, at the step S30C, the CPU 30 controls the driving circuit 14B to cause the light emitting element 1C to emit light with the light quantity Q₁. That is, in the standby mode, the light emitting element 1C is caused to emit light while the light emitting element 1A is prevented from emitting light.

Then, when the living body 8 is detected at the measurement position based on the spectral distribution of light received by the light receiving element 3 at the step S40 and the biometric information measuring apparatus 10B shifts from the standby mode to the measurement mode, the CPU 30 controls the driving circuit 14B to stop the emission of the light emitting element 1C at the step S55 and thereafter allow the light emitting element 1A to emit light with the light quantity Q₂ at the step S60.

Then, the CPU 30 performs a process after the step S60, which has been described in FIG. 9, to measure the biometric information.

In this way, with the biometric information measuring apparatus 10B according to the third exemplary embodiment, the dedicated light emitting element 1C whose wavelength of light is adjusted for detection of the living body 8 is used to detect the living body 8 and the light emitting element 1A provided exclusively for measurement of the biometric information is used to measure the biometric information.

Therefore, since the light emitting elements respectively suitable for the characteristics related to the detection of the living body 8 and the characteristics related to the measurement of the biometric information are used, it is expected to improve the detection accuracy of the living body 8 and the measurement accuracy of the biometric information as compared with the case where the same light emitting element 1A is used for detection of the living body 8 and measurement of the biometric information.

In addition, the biometric information measuring apparatus 10B sets the light quantity in the standby mode to be smaller than the light quantity in the measurement mode to reduce the light quantity with which the user may be unintentionally irradiated in the standby mode, thereby reducing a user's stress and power consumption caused by the irradiation of the user body with the light.

The contents and various modification examples suggested to the biometric information measuring apparatus 10 according to the first exemplary embodiment are also applied to the biometric information measuring apparatus 10B.

For example, after the user is notified that the living body 8 is separated from the measurement position in the step S90, the emission of the light emitting element 1A may be stopped and the process may proceed to the step S20 to return again to the standby mode.

Further, in order to notify the user whether the biometric information measuring apparatus 10B is in the standby mode or in the measurement mode, the biometric information measuring apparatus 10B may change the contents displayed on the display device 34 for each mode.

Further, the biometric information measuring apparatus 10B may stop the supply of power to the display device 34 in the standby mode and may start the supply of power to the display device 34 at the time of shifting to the measurement mode to display information on the display device 34.

Furthermore, as described in the first modification example of the first exemplary embodiment, the biometric information measuring apparatus 10B may allow the light emitting element 1C to emit light in the standby mode with the same light quantity Q₂ as the light emitting element 1A in the measurement mode.

Moreover, as described in the second modification example of the first exemplary embodiment, when the quantity of received light by external light is equal to or less than a received light quantity threshold value and the living body 8 is detected at the measurement position, the biometric information measuring apparatus 10B may shift from the standby mode to the measurement mode to measure the biometric information.

In the biometric information measuring apparatus 10B, as an example, one light emitting element 1A and one light emitting element 1C are included. However, for example, two or more light emitting elements 1A and two or more light emitting elements 1C may be included.

Although the present invention has been described above by way of some exemplary embodiments, the present invention is not limited to the scope described in the exemplary embodiments. Various modifications or improvements can be made to the exemplary embodiments without departing from the spirit and scope of the present invention and are included in the technical scope of the present invention. For example, without departing from the gist of the present invention, the order of processes may be changed or the present invention may be applied to measurement of a blood velocity in addition to the blood flow rate.

Further, as illustrated in FIG. 19, since the intensity of light received by the light receiving element 3 is varied depending on the pulsation of an artery, a pulse rate may be measured from a change in received light intensity in the light receiving element 3. Further, an acceleration pulse wave may be measured by twice differentiating a waveform obtained by measuring a change in the pulse rate in a chronological order. The acceleration pulse wave is used for estimation of a blood vessel age, diagnosis of arteriosclerosis, or the like. In this way, the present invention is not limited to the contents exemplified here but may be used to measure other biometric information.

Further, the above exemplary embodiments may be applied to a mobile terminal such as a wearable terminal. In this case, a user's action of wearing the terminal may be detected and used as an instruction to start measurement. For example, a sensor for detecting motion of a terminal, such as an acceleration sensor, may be mounted, and an instruction to start measurement may be issued when a predetermined motion of the terminal is detected.

In the above described exemplary embodiments, as an example, the processes in the controller 12 (12A and 12B), the detecting unit 20 (20A) and the measuring unit 22 (22A) are implemented by software. However, the same processes as the flowcharts illustrated in FIGS. 9, 15, 16, 21 and 25 may be implemented by hardware. This may increase a process speed as compared with the case where the processes in the controller 12 (12A and 12B), the detecting unit 20 (20A) and the measuring unit 22 (22A) are implemented by software.

Further, in the above described exemplary embodiments, the biometric information measuring program is installed in the ROM 31. However, the present invention is not limited thereto. The biometric information measuring program according to the exemplary embodiments of the present invention may be provided in a form recorded in a computer readable recording medium. For example, the biometric information measuring program according to the exemplary embodiments of the present invention may be provided in a form recorded in a portable recording medium such as a CD (Compact Disc)-ROM, DVD (Digital Versatile)-ROM, USB (Universal Serial Bus) memory or the like. Further, the biometric information measuring program according to the exemplary embodiments of the present invention may be provided in a form recorded in a semiconductor memory such as a flash memory or the like.

In addition, although the biometric information measuring apparatus 10 (10A and 10B) measures the biometric information after detecting the living body 8 using the spectral distribution of light emitted from the light emitting element(s), the above-described detecting unit for detecting the living body 8 may be applied to a living body detecting device that detects the presence or absence of the living body 8 at a specific position.

For example, the detecting unit for detecting the living body 8, which has been described in the exemplary embodiments, may be applied to a determination as to whether or not an object is worn on the user's body, a determination as to whether or not the living body 8 is present in a specific place or space, etc.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A biometric information measuring apparatus comprising:
a light emitting unit configured to emit light;
a light receiving unit configured to receive light;
a detecting unit configured to detect a frequency distribution of the light received by the light receiving unit; and
a controller, wherein when a feature which is obtained in response to a living body being irradiated with light is no longer included in a frequency component detected by the detecting unit during a period in which biometric information is measured using the frequency distribution detected by the detecting unit, the controller stops measurement of biometric information in the living body.

2. The biometric information measuring apparatus according to claim 1, wherein the controller controls the light emitting unit such that a quantity of light emitted from the light emitting unit during a period in which the measurement of the biometric information is stopped becomes smaller than a quantity of light emitted from the light emitting unit during a period in which the biometric information is measured.

3. The biometric information measuring apparatus according to claim 2, wherein the controller controls the light emitting unit to stop emission of the light emitting unit during the period in which the measurement of the biometric information is stopped.

4. The biometric information measuring apparatus according to claim 1, further comprising:
a notification unit configured to notify that the measurement of the biometric information is stopped when the measurement of the biometric information is stopped.

5. The biometric information measuring apparatus according to claim 1, wherein when a magnitude of a frequency component at a predetermined frequency in the frequency distribution detected by the detecting unit is equal to or smaller than a threshold value preset as a value which is obtained in response to the living body being irradiated with light, the controller stops the measurement of the biometric information in the living body.

6. The biometric information measuring apparatus according to claim 5, wherein the controller sets a plurality of predetermined frequencies for the frequency distribution detected by the detecting unit and stops the measurement of the biometric information in the living body when the magnitude of the frequency component at each of the plurality of predetermined frequencies is equal to or smaller than the threshold value.

7. The biometric information measuring apparatus according to claim 1, wherein if a quantity of light received by the light receiving unit is equal to or smaller than a predetermined received light quantity during a period in which light is not emitted from the light emitting unit and if the feature is included in the frequency distribution detected by the detecting unit during a period in which light is emitted from the light emitting unit, the controller controls an operation state of the apparatus to switch from a standby state to a measurement state in which the biometric information in the living body is measured.

8. The biometric information measuring apparatus according to claim 1, wherein the detecting unit detects the frequency distribution in a frequency region included in the light which is emitted from the light emitting unit and transmitted through a blood vessel of the living body or reflected by the blood vessel of the living body.

9. A biometric information measuring apparatus comprising:
a light emitting unit configured to emit light;
a light receiving unit configured to receive light;
a detecting unit configured to detect a magnitude of a frequency component in a frequency distribution of the light received by the light receiving unit; and
a controller, wherein when the magnitude of the frequency component is smaller than a magnitude of the frequency component which is obtained in response to a living body being irradiated with light during a measurement period in which biometric information is measured, the controller controls a state of the apparatus to switch to a state different from a state in the measurement period.

10. A non-transitory computer readable storage medium storing a biometric information measuring program, the program causing a computer to function as:
a detecting unit configured to detect a frequency distribution of received light; and
a controller, wherein when a feature which is obtained in response to a living body being irradiated with light is no longer included in a frequency component detected by the detecting unit during a period in which biometric information is measured using the frequency distribution detected by the detecting unit, the controller stops measurement of biometric information in the living body.
